SEQUENCE LISTING (17 sequences)

SEQ ID NO: 1 - DNA, 3258 bp, Artificial Sequence
  IBDV strain R903/78 segment A
ggatacgatcggtctgaccccgggggagtcacccggggacaggccgtcaaggctttgttccaggatggaactcctccttctacaacgctatcattgatggtcagtagagatcagacaaacgatcgcagcgatgacaaacctgcaagatcaaacccaacagattgttccgttcatacggagccttctgatgccaacaaccggaccggcgtccattccggacgacaccctggagaagcacactctcaggtcagagacctcgacctacaatttgactgtgggggacacagggtcagggctaattgtctttttccctggattccctggctcaattgtgggtgctcactacacactgcagagcaatgggaactacaagttcgatcagatgctcctgactgcccagaacctaccggccagttacaactactgcaggctagtgagtcggagtctcacagtgaggtcaagcacacttcctggtggcgtttatgcactaaacggcaccataaacgccgtgaccttccaaggaagcctgagtgaactgacagatgttagctacaatgggttgatgtctgcaacagccaacatcaacgacaaaattgggaacgtcctagtaggggaaggggtcaccgtcctcagcttacccacatcatatgatcttgggtatgtgaggcttggtgaccccattcccgcaatagggcttgacccaaaaatggtagccacatgtgacagcagtgacaggcccagagtctacaccataactgcagccgatgattaccaattctcatcacagtaccaaccaggtggggtaacaatcacactgttctcagccaacattgatgccatcacaagcctcagcgttgggggagagctcgtgtttcaaacaagcgtccacggcattgtactgggcgccaccatctacctcataggctttgatgggacagcggtaatcaccagggctgtggccgcaaacaatgggctgacgaccggcaccgacaaccttatgccattcaatcttgtgattccaacaaacgagataacccagccaatcacatccatcaaactggagatagtgacctccaaaagtggtggtcaggcaggggatcagatgtcatggtcggcaagagggagcctagcagtgacgatccatggtggcaactatccaggggccctccgtcccgtcacgctagtggcctacgaaagagtggcaacaggatccgtcgttacggtcgctggggtgagcaacttcgagctgatcccaaatcctgaactagcaaagaacctggttacagaatacggccgatttgacccaggagccatgaactacacaaaattgatactgagtgagagggaccgtcttggcatcaagaccgtctggccaacaagggagtacactgactttcgtgaatacttcatggaggtggccgacctcaactctcccctgaagattgcaggagccttcggcttcaaagacataatccgggccataaggaggatagctgtgccggtggtctccacattgttcccacctgccgctcccctagcccatgcaattggggaaggtgtagactacctgctgggcgatgaggcacaggctgcttcaggaactgctcgagccgcgtcaggaaaagcaagagctgcctcaggccgcataaggcagctgactctcgccgccgacaaggggtacgaggtagtcgcgaatctattccaggtgccccagaatcccgtagtcgacgggattcttgcttcacctggggtactccgcggtgcacacaacctcgactgcgtgttaagagagggtgccacgctattccctgtggttattacgacagtggaagacgccatgacacccaaagcattgaacagcaaaatgtttgctgtcattgaaggcgtgcgagaagacctccaacctccatctcaaagaggatccttcatacgaactctctctggacacagagtctatggatatgctccagatggggtacttccactggagactgggagagactacaccgttgtcccaatagatgatgtctgggacgacagcattatgctgtccaaagatcccatacctcctattgtgggaaacagtggaaatctagccatagcttacatggatgtgtttcgacccaaagtcccaatccatgtggctatgacgggagccctcaatgcttgtggcgagattgagaaagtaagctttagaagcaccaagctcgccactgcacaccgacttggccttaagttggctggtcccggagcattcgatgtaaacaccgggcccaactgggcaacgttcatcaaacgtttccctcacaatccacgcgactgggacaggctcccctacctcaacctaccataccttccacccaatgcaggacgccagtaccaccttgccatggctgcatcagagttcaaagagacccccgaactcgagagtgccgtcagagcaatggaagcagcagccaacgtggacccactattccaatctgcactcagtgtgttcatgtggctggaagagaatgggattgtgactgacatggccaacttcgcactcagcgacccgaacgcccatcggatgcgaaattttcttgcaaacgcaccacaagcaggcagcaagtcgcaaagggccaagtacgggacagcaggctacggagtggaggctcggggccccacaccagaggaagcacagagggaaaaagacacacggatctcaaagaagatggagaccatgggcatctactttgcaacaccagaatgggtagcactcaatgggcaccgagggccaagccccggccagctaaagtactggcagaacacacgagaaataccggacccaaacgaggactatctagactacgtgcatgcagagaagagccggttggcatcagaagaacaaatcctaagggcagctacgtcgatctacggggctccaggacaggcagagccaccccaagctttcatagacgaagttgccaaagtctatgaaatcaaccatggacgtggcccaaaccaagaacagatgaaagatctgctcttgactgcgatggagatgaagcatcgcaatcccaggcgggctctaccaaagcccaagccaaaacccaatgctccaacacagagaccccctggtcggctgggccgctggatcaggaccgtctctgatgaggaccttgagtgaggctcctgggagtctcccgacaccacccgcgcaggtgtggacaccaattcggccttacaacccaaattggatccgttcgcgggtcccct

SEQ ID NO: 2 - DNA, 2827 bp, Artificial Sequence
  IBDV strain R903/78 segment B
ggatacgatgggtttgaccctcagggagtcacgaattaacatggctactaggggcgatacccgccgctggctgccacgttagtggctcctcttcttgatgattctgccaccatgagtgacattttcaacagtccacaggcgcgaagcacgatctcagcagcgttcggcataaagcctactgctggacaagacgtggaagaactcttgatccctaaagtttgggtgccacctgaggatccgcttgccagccctagtcgactggcaaagttcctcagagagaacggctacaaagttttgcagccacggtctctgcccgagaatgaggagtatgagaccgaccaaatactcccagacttagcatggatgcgacagatagaaggggctgttttaaaacccactctatctctccctattggagatcaggagtacttcccaaagtactacccaacacatcgccctagcaaggagaagcccaatgcgtacccgccagacatcgcactactcaagcagatgatttacctgtttctccaggttccagaggccaacgagggcctaaaggatgaagtaaccctcttgacccaaaacataagggacaaggcctatggaagtgggacctacatgggacaagcaactcgacttgtggccatgaaggaggtcgccactggaagaaacccaaacaaggatcctctaaagcttgggtacacttttgagagcatcgcgcagctacttgacatcacactaccggtaggcccacccggtgaggatgacaagccctgggtgccactcacaagagtgccgtcacggatgttggtgctgacgggagacgtagatggcgactttgaggttgaagattaccttcccaaaatcaacctcaagtcatcaagtggactaccatatgtaggtcgcaccaaaggagagacaattggcgagatgatagctatctcaaaccagtttctcagagagctatcaacactgttgaagcaaggtgcagggacaaaggggtcaaacaagaagaagctactcagcatgttaagtgactattggtacttatcatgcgggcttttgtttccaaaggctgaaaggtacgacaaaagcacatggctcaccaagacccggaacatatggtcagctccatccccaacacacctcatgatctctatgatcacctggcccgtgatgtccaacagcccaaataacgtgttgaacattgaagggtgtccatcactctacaaattcaacccgttcagaggagggttgaacaggatcgtcgagtggatattggccccggaagaacccaaggctcttgtatatgcggacaacatatacattgtccactcaaacacgtggtactcaattgacctagagaagggtgaggcaaactgcactcgccaacacatgcaagccgcaatgtactacatactcaccagagggtggtcagacaacggcgacccaatgttcaatcaaacatgggccacctttgccatgaacattgcccctgctctagtggtggactcatcgtgcctgataatgaacctgcaaattaagacctatggtcaaggcagcgggaatgcagccacgttcatcaacaaccacctcttgagcacgctagtgcttgaccagtggaacctgatgagacagcccagaccagacagcgaggagttcaaatcaattgaggacaagctaggtatcaactttaagattgagaggtccattgatgatatcaggggcaagctgagacagcttgtcctccttgcacaaccagggtacctgagtgggggggttgaaccagaacaatccagcccaactgttgagcttgacctactagggtggtcagctacatacagcaaagatctcgggatctatgtgccggtgcttgacaaggaacgcctattttgttctgctgcgtatcccaagggagtagagaacaagagtctcaaatccaaagtcgggatcgagcaggcatacaaggtagtcaggtatgaggcgttgaggttggtaggtggttggaactacccactcctgaacaaagcctgcaagaataacgcaggcgccgctcggcggcatctggaggccaaggggttcccactcgacgagttcctagccgagtggtctgagctgtcagagttcggtgaggccttcgaaggcttcaatatcaagctgaccgtaacatctgagagcctagccgaactgaacaagccagtaccccccaagcccccaaatgtcaacagaccagtcaacactgggggactcaaggcagtcagcaacgccctcaagaccggtcggtacaggaacgaagccggactgagtggtctcgtccttctagccacagcaagaagccgtctgcaagatgcagttaaggccaaggcagaagccgagaaactccacaagtccaagccagacgaccccgatgcagactggttcgaaagatcagaaactctgtcagaccttctggagaaagccgacatcgccagcaaggtcgcccactcagcactcgtggaaacaagcgacgctcttgaagcagttcagtcgacttccgtgtacacccccaagtacccagaagtcaagaacccacagaccgcctccaaccccgttgttgggctccacctgcccgccaagagagccaccggtgtccaggccgctcttctcggagcaggaacgagcagaccaatggggatggaggccccaacacggtccaagaacgccgtgaaaatggccaaacggcggcaacgccaaaaggagagccgctaacagccatgatgggaaccactcaagaagaggacactaatcccagaccccgtatccccggccttcgcctgcgggggccccc

SEQ ID NO: 3 - DNA, 20 bp, Artificial Sequence
  IBDV_f primer
tcactacacactgcagagca

SEQ ID NO: 4 - DNA, 20 bp, Artificial Sequence
  IBDV_r primer
gagactccgactcactagcc

SEQ ID NO: 5 - DNA, 20 bp, Artificial Sequence
  IBDV_p
tgcccagaacctaccggcca

SEQ ID NO: 6 - DNA, 20 bp, Artificial Sequence
  IFN-beta_f primer
gacgccgcattgaccatcta

SEQ ID NO: 7 - DNA, 22 bp, Artificial Sequence
  IFN-beta_r primer
ccttaggatttccactctgact

SEQ ID NO: 8 - DNA, 21 bp, Artificial Sequence
  IFN-lambda_f primer
tgagctggccctgacgctgaa

SEQ ID NO: 9 - DNA, 19 bp, Artificial Sequence
  IFN-lambda_r primer
aggcggaggttgaaggtga

SEQ ID NO: 10 - DNA, 28 bp, Artificial Sequence
  IFN-gamma_f primer
gaaaagctgactaattattcggtaactg

SEQ ID NO: 11 - DNA, 22 bp, Artificial Sequence
  IFN-gamma_r primer
gttcagccatcacttggatgag

SEQ ID NO: 12 - DNA, 21 bp, Artificial Sequence
  ISG56_f primer
cctggagtactatgagcgggc

SEQ ID NO: 13 - DNA, 21 bp, Artificial Sequence
  IFG56_r primer
tgggtgcctaaggaccttgtc

SEQ ID NO: 14 - DNA, 20 bp, Artificial Sequence
  MxA_f primer
aggtcagttaccaggactac

SEQ ID NO: 15 - DNA, 20 bp, Artificial Sequence
  MxA_r primer
atggcattctgggctttatt

SEQ ID NO: 16 - DNA, 23 bp, Artificial Sequence
  GAPDH_f primer
ggtatcgtggaaggactcatgac

SEQ ID NO: 17 - DNA, 23 bp, Artificial Sequence
  GAPDH_r primer
atgccagtgagcttcccgttcag

(19) United States
(12) Patent Application Publication
(10) Pub. No.: US 2024/0024390 A1
(43) Pub. Date: Jan. 25, 2024

(54) USE OF A BIRNAVIRUS ALONE OR IN COMBIMATION THERAPY FOR THE TREATMENT OF A DISEASE CAUSED BY A NIDOVIRUS

(71) Applicant: Probiogen AG, Berlin (DE)

(72) Inventors: Imre Kovesdi, Rockville, MD (US); Tibor Bakacs, Budapest (HU); Volker Sandig, Berlin (DE); Shimon Slavin, Tel Aviv (IL); Deborah Horn, Berlin (DE); Alexander Karlas, Berlin (DE); Sven Krugener, Berlin (DE)

(21) Appl. No.: 18/258,386

(22) PCT Filed: Dec. 21, 2020
(86) PCT No.: PCT/EP2020/087550

§ 371 Date: Jun. 20, 2023

(51) Int. Cl. A61K 35/76; C12N 7/00; A61K 45/06; A61J 1/05; A61J 1/20; A61P 31/14

(52) U.S. Cl. CPC A61K 35/76; C12N 7/00; A61K 45/06; A61J 1/05; A61J 1/20; A61P 31/14

(57) ABSTRACT

The present invention relates to a birnavirus for use in the treatment of a disease caused by a nidovirus. Further, the present invention relates to a combination comprising at least one birnavirus and at least one further active agent for use in the treatment of a disease caused by a nidovirus. Furthermore, the present invention relates to a pharmaceutical composition comprising the birnavirus or the combination for use in the treatment of a disease caused by a nidovirus.

The present invention relates to a birnavirus for use in the treatment of a disease caused by a nidovirus. Further, the present invention relates to a combination comprising at least one birnavirus and at least one further active agent for use in the treatment of a disease caused by a nidovirus. Furthermore, the present invention relates to a pharmaceutical composition comprising the birnavirus or the combination for use in the treatment of a disease caused by a nidovirus.

### BACKGROUND OF THE INVENTION

The order Nidovirales comprises four families (Coronaviridae, Arteriviridae, Roniviridae and Mesoniviridae) of viruses with a non-segmented, single-stranded RNA genome of positive polarity (ssRNA). Nidoviruses infect a broad range of hosts including humans and other mammals, birds, fish, insects, and crustaceans. They cause important infections in mammals. Especially coronaviruses cause about 30% of common colds in humans. Although upper and lower respiratory tract manifestations are the most commonly reported symptoms in coronavirus caused diseases, the gastrointestinal tract and liver might also be affected. As nidoviruses are known to mutate and recombine often, they present an ongoing challenge for disease control.

An outbreak of the novel zoonotic severe acute respiratory syndrome coronavirus 2 (SARS-CoV-2) in November 2019 spread within several weeks around the world leading to approximately 68 million infections up to date (December 2020) and more than 1,557,000 deaths. The economic impact of the Coronavirus Disease 2019 (COVID-19) pandemic is enormous, primarily because of the required quarantine measures to restrict the uncontrolled spreading of the virus.

In order to mitigate the impact of the COVID-19 outbreak, therapeutic approaches are needed to complement the development of prophylactic vaccines. As with many other viral pathogens, available treatment involves supportive measures such as anti-pyretics to keep fever down, fluids, antibiotics for secondary bacterial infections and respiratory support as necessary. In addition to individual healing attempts with substances that have not yet been approved, medicines which are also approved for other diseases are used in therapeutic trials in COVID-19 patients. This approach takes place in what is known as “off-label use”, i.e. the drugs used are generally approved the respective country and therefore marketable under pharmaceutical law but not used in the approved indication, population and/or dosage.

Among antiviral drugs, only Remdesivir (Veklury®) has so far been approved for application in COVID-19 via a conditional admission. Remdesivir is used to treat COVID-19 in adults and adolescents with pneumonia that requires an additional supply of oxygen. According to the interim results (October 2020) of the “Solidarity” study of the World Health Organization (WHO), which also tests chloroquine/hydroxychloroquine, lopinavir/ritonavir (some in combination with beta interferon), no or at most only minor healing effects could be demonstrated by Remdesivir.

Another therapeutic approach is the use of convalescent plasma. The use in various other viral infections (e.g. by SARS-CoV or MERS-CoV) led to different results. The effectiveness of convalescent plasma is therefore discussed critically and divergently in the literature. Several therapy studies have been initiated. So far, the evidence is insufficient for a recommendation.

In some of the patients a situation develops in the course of the disease, which resembles secondary, virus-triggered hemophagocytic lymphohistiocytosis (sHLH, “Cytokine storm”). These patients show massive inflammation, mostly high fever significantly increased IL-6 and ferritin levels. As a possible therapy approach in this situation a blockade of the interleukin-6 (IL-6) receptor e.g. with tocilizumab, sarilumab or siltuximab is discussed.

However, there is still an unmet need to provide therapeutic compounds and methods for treating a subject infected with a nidovirus in general and a coronavirus like SARS-CoV-2 in particular. Especially, there is still an unmet need for effective, inexpensive, easy to administer, transport and storage stable and safe therapeutics for therapy of diseases caused by a nidovirus.

The present inventors surprisingly found that viruses of the Birnaviridae family can be used as medicament, specifically as medicament for therapy of diseases caused by a nidovirus such as coronavirus like SARS-CoV-2. In particular, the present inventors surprisingly found that viruses of the Birnaviridae family promote activation of the interferon-dependent antiviral gene program and, thus, allow an efficient treatment of a broad range of different nidovirus infections/diseases.

Birnaviridae are small (70 nm in diameter), non-enveloped viruses, with segmented, linear double-stranded RNA (dsRNA) genome which codes for 5 to 6 proteins in segments A and B. Birds, fishes, and insects serve as natural hosts. Infectious bursal disease virus (IBDV) is currently the only species within the Avibirnavirus genus. IBDV is a common poultry pathogen causing the infectious bursal disease (IBD), which is also known as Gumboro disease.

It infects B cell precursors in young chickens. Vaccine strains are safely used in mass vaccinations for 50+ years.

By using the clinically proven attenuated live avian virus IBDV R903/78 in human, the present inventors surprisingly found that a pharmaceutical composition containing between 10⁶ and 10⁹ infectious units per dose is effective in the treatment of a disease caused by a nidovirus in humans and safe for humans. They further surprisingly found that a pharmaceutical composition comprising attenuated live avian virus IBDV R903/78 is suitable for the treatment of COVID-19. These findings show that viruses of the Birnaviridae family have the properties for passive immunotherapy (e.g. via interferon induction initiated by the dsRNA of an apathogenic virus). A medicament comprising a virus of the Birnaviridae family is therapeutically efficient with oral application in upper and lower respiratory infections of nidoviruses, but also in infections not exposed to the respiratory tract and the upper gastrointestinal tract. According to the present inventors, the virus of the Birnaviridae family can be used in a monotherapy or in a combination therapy to treat infections caused by a nidovirus, preferably coronavirus, more preferably SARS-CoV-2.

### SUMMARY OF THE INVENTION

In a first aspect, the present invention relates to a birnavirus for use in the treatment of a disease caused by a nidovirus.

In a second aspect, the present invention relates to a combination comprising at least one birnavirus and at least one further active agent for use in the treatment of a disease caused by a nidovirus.

In a third aspect, the present invention relates to a pharmaceutical composition comprising a birnavirus as defined in the first aspect or a combination as defined in the second aspect for use in the treatment of a disease caused by a nidovirus.

In a fourth aspect, the present invention relates to a reservoir comprising a birnavirus as defined in the first aspect, a combination as defined in the second aspect, or a pharmaceutical composition as defined in the third aspect, wherein the reservoir is designed to be inserted into an oral or a nasal applicator.

In a fifth aspect, the present invention relates to an oral or nasal applicator comprising
(i) a reservoir comprising a birnavirus as defined in the first aspect, a combination as defined in the second aspect, or a pharmaceutical composition as defined in the third aspect, and
(ii) a mouth piece or nose piece, wherein the mouth piece or the nose piece is connected with/attached to the reservoir.

In a sixth aspect, the present invention relates to a kit comprising
(i) a packaging material, and
(ii) a birnavirus as defined in the first aspect, a combination as defined in the second aspect, a pharmaceutical composition as defined in the third aspect, a reservoir of the fourth aspect, and/or an oral or nasal applicator of the fifth aspect.

In a seventh aspect, the present invention relates to a birnavirus for use as medicament, wherein the birnavirus is to be administered to a subject orally, nasally, or by inhalation.

In an eight aspect, the present invention relates to a pharmaceutical composition comprising a birnavirus as defined in the first aspect or a combination as defined in the second aspect, wherein the composition is in a form suitable for oral administration, nasal administration, or administration by inhalation.

This summary of the invention does not describe all features of the invention.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. As used in this specification and the appended claims, the singular forms “a”, “an”, and “the” include plural referents, unless the content clearly dictates otherwise.

The term “consisting essentially of”, as used herein, limits the scope of a claim to the specified materials or steps and those that do not materially affect the basic and novel characteristic(s) of the claimed invention. In other words, the term “consisting essentially of”, as used herein, is generally construed to mean that the composition or formulation (a) necessarily includes the listed ingredients and (b) is open to unlisted ingredients that do not materially affect the basic and novel properties of the composition. Similarly, when “consisting essentially of” is used herein in a process claim, the claim requires that the listed steps are performed, but also may include unlisted steps that do not affect the basic and material properties of the process.

The term “about” when used in connection with a numerical value is meant to encompass numerical values within a range having a lower limit that is 5% smaller than the indicated numerical value and having an upper limit that is 5% larger than the indicated numerical value.

In the context of the present invention, a birnavirus is used as a medicament, in particular for the treatment of a disease caused by a nidovirus. The term “birnavirus”, as used herein, refers to a small (about 70 nm in diameter), non-enveloped virus. It is a segmented, linear, double-stranded (ds) RNA virus. The genome is about 5.9 to 6.9 kbp in length and codes for 5 to 6 proteins in segments A (Genebank Accession Number: JQ411012.1) and B (Genebank Accession Number: JQ411013.1). Birds, fishes, and insects are described as natural hosts. The birnavirus replication is cytoplasmic. Entry into the host cell is achieved by cell receptor endocytosis. Replication follows the double-stranded RNA virus replication model in the cytoplasm. Double-stranded RNA virus transcription is the method of transcription in cytoplasm. A birnavirus typically releases its progeny via continuous budding of viral particles from an intact cell membrane.

In one preferred embodiment, the birnavirus is selected from the group consisting of an avibirnavirus, an aquabirnavirus, a blosnavirus, a dronavirus, an entomobirnavirus, a ronavirus, and a telnavirus. In one more preferred embodiment, the avibirnavirus is an Infectious Bursal Disease Virus (IBDV). In one even more preferred embodiment, the IBDV is an IBDV of strain 903/78. In one still even more preferred embodiment, the (genome of) IBDV of strain 903/78 comprises a nucleotide sequence according to SEQ ID NO: 1 (segment A), a fragment thereof or a sequence having at least 80% sequence identity thereto, and/or a nucleotide sequence according to SEQ ID NO: 2 (segment B), a fragment thereof or a sequence having at least 80% sequence identity thereto.

In particular, the nucleotide sequence can be selected from the group consisting of
(i) a nucleotide sequence according to SEQ ID NO: 1 or SEQ ID NO: 2,
(ii) a nucleotide sequence that is a fragment of the nucleotide sequence according to (i), preferably a nucleotide sequence that is a fragment which is between 1 and 12, more preferably between 1 and 8, and most preferably between 1 and 5 or 1 and 3, i.e. 1, 2, 3, 4, 5, 6, 7, 8, 9, 10, 11, or 12, nucleotides shorter than the nucleotide sequence according to (i), and
(iii) a nucleotide sequence that has at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

The similarity of nucleotide sequences, i.e. the percentage of sequence identity, can be determined via sequence alignments. Such alignments can be carried out with several art-known algorithms, preferably with the mathematical algorithm of Karlin and Altschul (Karlin & Altschul (1993) Proc. Natl. Acad. Sci. USA 90:5873-5877), with hmmalign (HMNIER package, http://hmmer.wustl.edu/) or with the CLUSTAL algorithm (Thompson J. D. et al. Nucleic Acids Res. 1994, 22:4673-80) available e.g. on http://www.ebi.ac.uk/Tools/clustalw/ or on http://www.ebi.ac.uk/Tools/clustalw2/index.html or on http://npsa-pbil.ibcp.fr/cgi-bin/npsa_automat.pl?page=/NPSA/npsa_clustalw.html. Preferred parameters used are the default parameters as they are set on http://www.ebi.ac.uk/Tools/clustalw/ or http://www.ebi.ac.uk/Tools/clustalw2/index.html. The grade of sequence identity (sequence matching) may be calculated using e.g. BLAST, BLAT or BlastZ (or BlastX). A similar algorithm is incorporated into the BLASTN and BLASTP programs of Altschul et al. J. Mol. Biol. 1990, 215:403-410. To obtain gapped alignments for comparative purposes, Gapped BLAST is utilized as described in Altschul et al. Nucleic Acids Res. 1997, 25:3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields.

The birnavirus of the present invention is apathogenic (i.e. not capable of causing disease) in humans and in the respective natural host.

As mentioned above, a birnavirus is used for the treatment of a disease caused by a nidovirus. The term “nidovirus”, as used herein, refers to a positive sense, single-stranded RNA ((+) ssRNA) enveloped virus. The genomic RNA of nidoviruses is infectious. Nidoviruses enter the cell via specific receptors.

One example of a nidovirus is a coronavirus. Certain coronaviruses, for example, enter the cell via the angiotensin-converting enzyme 2 (ACE2) receptor. This is a cellular receptor which is expressed ubiquitously on endothelia, e.g. in the lungs, arteries, heart, kidneys, and the intestine. ACE2 binds to the viral (S) protein and allows the cellular entry. More specifically, the S protein is cleaved into two subunits, S1 and S2, by an extracellular protease. While 51 binds to ACE2, S2 is further cleaved and activated by the host surface-associated transmembrane protease serine 2 (TMPRSS2). Together these actions result in host-viral membrane fusion and the release of the RNA genome into the host cell cytoplasm.

Upton entry into the host cell, the translational machinery of the host is hijacked for the translation of the two replicase polyproteins (pp1a and pp1ab) and the essential viral proteases (3CLpro and PLpro). The two replicase polyproteins (pp1a and pp1ab) are then cleaved into 16 non-structural effector proteins by the viral proteases (3CLpro and PLpro) allowing them to form the replication complex together with the RNA-dependent RNA polymerase, which synthesizes a full-length negative RNA strand template. This is used to replicate the complete RNA genome and generate the individual subgenomic mRNA templates needed for the translation of the viral structural and accessory proteins. More specifically, the mRNAs for the genes downstream of ORF 1a/1b share a 5′ leader and a body consisting of the respective first and all subsequent ORFs. In this way, the replicase first transcribes subgenomic RNAs with genomic RNA as template. Initiating at the 3′ UTR, transcription proceeds towards an element termed transcription regulating sequence (TRS-B) upstream of each gene in the body of the genome (the region downstream of ORF 1a/1b). Homology of the TRS-B to a corresponding element in the 5′ UTR (TRS-L) promotes a template switch so that the common leader of all viral mRNAs is being obtained. Out of these different antisense instances, the mRNAs are next transcribed as plus-strand copies. Translation occurs at the rough endoplasmic reticulum (ER). The translated structural and accessory proteins are then trafficked from the ER through the Golgi apparatus, after which new virions assemble in budding Golgi vesicles. The packaging signal of nidoviruses is located in the 3′ half of ORF 1b. After packaging, the mature virions are exocytosed and released from the host cell into the surrounding environment to repeat the infection cycle.

Predominant hosts of the nidoviruses are mammals and birds (Coronaviridae and Arterivirdae), crustaceans (Roniviridae) and mosquitos (Mesoniviridae). Important pathogens in the Arteriviridae are viruses that cause porcine reproductive and respiratory syndrome (PRRS) and equine viral arteritis (EVA).

In one preferred embodiment, the nidovirus is a virus of the family of Coronaviridae, Arteriviridae, Roniviridae, or Mesoniviridae. In one more preferred embodiment, the nidovirus is a virus of the family of Coronaviridae or Arteriviridae. Within the Coronaviridae, one subfamily is termed Orthocoronavirinae and the four genera therein are alpha-coronavirus, beta-coronavirus, delta-coronavirus and gamma-coronavirus. The alpha- and beta-coronaviruses appear to have evolved in bat reservoirs and the delta- and gamma-coronaviruses appear to have evolved in avian reservoirs. The coronavirus may be an alpha-coronavirus or a beta-coronavirus. Examples of alpha-coronaviruses are HCoV-229E and CoV-NL63, and examples of beta-coronaviruses are SARS-CoV, SARS-CoV-2, MERS-CoV, HCoV-HKU1, and HCoV-OC43.

In one even more preferred embodiment, the virus of the family of Coronaviridae is selected from the group consisting of MERS-CoV, SARS-CoV, SARS-CoV-2, a coronavirus that gains entry by the ACE2 receptor, and an Infectious Bronchitis Virus (IBV). In one alternative even more preferred embodiment, the virus of the family of Arteriviridae is selected from the group consisting of a virus that causes porcine reproductive and respiratory syndrome (PRRS) and a virus that causes equine viral arteritits (EVA).

As mentioned above, a birnavirus is used for the treatment of a disease caused by a nidovirus. According to the present invention, the term “disease” refers to any pathological state caused by a nidovirus. The terms “disease” and “disorder” can be used interchangeable herein. The disease caused by a nidovirus is preferably a respiratory disease. The disease caused by a nidovirus is more preferably a severe acute respiratory syndrome (SARS). SARS is usually caused by a coronavirus. In particular, the coronavirus is severe acute respiratory syndrome coronavirus 2 (SARS-CoV-2).

As mentioned above, nidoviruses infect a broad range of hosts including humans and other mammals, birds, fish, insects and crustaceans. For example, coronaviruses cause about 30% of common colds in humans and, unlike rhinoviruses, cause both upper and lower respiratory infections, the latter being a more serious affliction. In addition, coronaviruses cause gastroenteritis and diarrhea in humans and many other serious diseases in non-human animals including mice, chickens, pigs, camels and cats.

A nidovirus, e.g. coronavirus, infection is defined by the entry of nidovirus, e.g. coronavirus, into at least one cell of a subject and its replication in the at least one cell. The infection can be an infection of the respiratory tract, including the upper respiratory tract (nose and nasal passages, paranasal sinuses, the pharynx, and the portion of the larynx above the vocal folds (cords)) and/or the lower respiratory tract (portion of the larynx below the vocal folds, trachea, bronchi, bronchioles and the lungs including the respiratory bronchioles, alveolar ducts, alveolar sacs and alveoli). The infection can also be characterized immunologically by the presence of at least one nidovirus-antigen-specific immune factor, preferably selected from the group consisting of B cells, follicular helper T cells (TFH cells), activated CD4+ T cells and CD8+ T cells (particularly also CD38+HLA-DR+), IgM antibodies, and IgG antibodies. Alternatively or additionally, it can be characterized immunologically by a nidovirus-specific cytokine profile.

A nidovirus infection may or may not cause symptoms of a nidovirus disease in a subject. The terms “nidovirus infection” and “nidovirus disease” are distinguished herein by the presence of at least one nidovirus disease symptom. As long as the infection is not accompanied by at least one symptom of nidovirus disease, it (or the subject) is asymptomatic (includes presymptomatic). The term “nidovirus disease” as used herein requires the presence of a nidovirus infection and at least one symptom of nidovirus disease (also referred to herein as “symptomatic infection”).

Nidovirus symptoms include dry cough, fever (≥37.8° C.), runny and/or blocked nose, fatigue, breathing difficulty, pneumonia, organ (e.g. heart, lung, liver and/or kidney) failure, itchy throat, headache, joint pain, nausea, diarrhoea, shivering, lymphophenia, loss of smell and/or loss of taste. Preferably, the nidovirus disease is characterized by the presence of two or more, three or more, or four or more symptoms, preferably including one or two or more of dry cough, fever (≥37.8° C.), breathing difficulty, loss of smell and/or loss of taste. The breathing difficulties may require intensive medical care including artificial ventilation. Nidoviruses are mainly transmitted by infected persons when they cough or sneeze. Protection is possible by washing hands frequently and avoiding touching the face. Also keeping a distance from people who feel unwell helps. Someone can also get infected by touching surfaces or things where the virus is present and then touching eyes, nose or mouth.

A nidovirus disease can occur in a mild form (no severe symptoms), in a moderate form, or in a severe form. For example, about 80% of the COVID-19 diseases are mild to moderate. 14% of the COVID-19 diseases are severe and in 6% % of the COVID-19 cases the clinical course is critical to life-threatening (with lung failure, septic shock, or multiple organ failure).

The term “mild form of nidovirus disease”, as used herein, refers to a nidovirus disease, e.g. coronavirus disease such as COVID-19, without signs of pneumonia. Mild symptoms are selected from the group consisting of dry cough, mild fever (≥37.8° C. to <40° C.), runny and/or blocked nose, fatigue, itchy throat, headache, joint pain, nausea, diarrhoea, shivering, lymphophenia, loss of smell, and loss of taste. In the mild form of nidovirus disease, e.g. coronavirus disease such as COVID-19, no respiratory distress is observed.

The term “moderate form of nidovirus disease”, as used herein, refers to a nidovirus disease, e.g. coronavirus disease such as COVID-19, associated with a mild pneumonia, which is radiologically limited to less than half of the lungs and an oxygen saturation in the blood of over 93%. Also, in the moderate form of nidovirus disease, e.g. coronavirus disease such as COVID-19, no respiratory distress is observed.

The term “severe form of nidovirus disease”, as used herein, refers to a nidovirus disease, e.g. coronavirus disease such as COVID-19, associated with breathing difficulty, in particular acute respiratory distress syndrome. The severe form of nidovirus disease, e.g. coronavirus disease such as COVID-19, is also associated with pneumonia and high fever (≥40° C.). The severe form of a nidovirus disease, e.g. coronavirus disease such as COVID-19, can be life-threatening. A life-threatening form of a nidovirus disease, e.g. coronavirus disease such as COVID-19, is usually associated with septic shock and/or multiple organ (e.g. heart, lung, liver and/or kidney) failure.

In one particularly preferred embodiment, the nidovirus disease, e.g. coronavirus disease such as COVID-19, is a mild or moderate form of a nidovirus disease, e.g. coronavirus disease such as COVID-19, i.e. a nidovirus disease, e.g. coronavirus disease such as COVID-19, not associated with an acute respiratory distress syndrome.

The term “Coronavirus Disease 2019 (COVID-19)”, as used herein, refers to an infectious disease caused by a nidovirus. In particular, it is caused by severe acute respiratory syndrome coronavirus 2 (SARS-CoV-2). The disease is a respiratory disease (similar to influenza) with symptoms such as cough and fever. Less common symptoms include fatigue, respiratory sputum production (phlegm), loss of the sense of smell, shortness of breath, muscle and joint pain, sore throat, headache, chills, vomiting, hemoptysis, diarrhea, or cyanosis. In more severe cases, breathing difficulties may occur. The breathing difficulties may require intensive medical care including artificial ventilation. COVID-19 is mainly transmitted by infected persons when they cough or sneeze. Protection is possible by washing hands frequently and avoiding touching the face. Also keeping a distance from people who feel unwell helps. Someone can also get infected by touching surfaces or things where the virus is present and then touching eyes, nose or mouth.

The term “live virus” as used herein, refers to a virus that is capable of multiplying and producing progeny virus upon infection of a permissive isolated cell or a permissive cell as part of an organism. Such cell may be permissive by nature or acquire permissiveness via introduction of functional sequences required for replication or mutation/deletion of sequences that would otherwise prevent multiplication.

The term “attenuated virus”, as used herein, refers to a virus with compromised virulence in the intended recipient, e.g. subject as defined herein. The birnavirus described herein is preferably an attenuated virus and more preferably a live and an attenuated virus. In particular, the birnavirus described herein is a replication competent live and attenuated virus.

The term “treatment”, in particular “therapeutic treatment”, as used herein, refers to any therapy which improves the health status and/or prolongs (increases) the lifespan of a subject suffering from a disease caused by a nidovirus such as coronavirus like SARS-CoV-2. Said therapy may eliminate the disease in a subject, arrest or slow the development of the disease in a subject, inhibit the development of the disease in a subject, decrease the severity of symptoms in a subject suffering the disease, and/or decrease the recurrence in a subject who currently has or who previously has had a disease.

The present invention relates to the use of a birnavirus as medicament. The term “medicament”, as used herein, refers to a substance/combination/composition used in therapy, i.e. in treating, ameliorating or preventing a disease or disorder. In particular, the present invention relates to the use of a birnavirus in the treatment of a disease caused by a nidovirus.

The treatment of a disease caused by a nidovirus, e.g. corona disease like COVID-19, described herein includes the administration of a birnavirus (monotherapy) or the administration of a birnavirus in combination with an additional drug (combination therapy). The drug is preferably selected from the group consisting of an antiviral drug, an antibacterial drug, an anti-inflammatory drug, an antifungal drug, an antipyretic agent, and a corticoid. The antiviral drug is more preferably selected from the group consisting of chloroquine, remdesivir, darunavir, favipiravir, lopinavir, and ritonavir. Combinations of these drugs are also encompassed. The antiviral drug is even more preferably selected from the group consisting of remdesivir, darunavir, or a combination of lopinavir and ritonavir. The antibacterial drug is more preferably selected from the group consisting of ceftriaxone, amoxicillin/clavulanic acid, piperacillin/tazobactam, and azithromycin. Combinations of these drugs are also encompassed. The anti-inflammatory drug is more preferably selected from the group consisting of ibuprofen and metamizole. Combinations of these drugs are also encompassed. The antifungal drug is more preferably selected from the group consisting of voriconazole and isavuconazole. Combinations of these drugs are also encompassed. The antipyretic agent is more preferably paracetamol. The corticoid is more preferably dexamethasone. However, also another treatment than the administration of a drug is possible. The other form of therapeutic treatment is preferably ventilation, especially when breath difficulties occur. More preferably, the ventilation is selected from the group consisting of non-invasive ventilation and invasive ventilation. Even more preferably, the non-invasive ventilation is carried out through a face mask, nasal mask, or a helmet, or the invasive ventilation is a mechanic ventilation, e.g. carried out through a positive pressure ventilator or negative pressure ventilator.

The term “monotherapy”, as used herein, refers to any therapy with only one active substance. In the context of the present invention, the active substance used in monotherapy to treat a disease caused by a nidovirus is a birnavirus. The term “combination therapy”, as used herein, refers to any therapy with two or more active substances. In the context of the present invention, the active substances used in combination therapy to treat a disease caused by a nidovirus are at least one birnavirus and at least one further active agent. The advantage of monotherapy is the possibility of a more targeted treatment of a disease, whereas combination therapy is intended to have a broader effect and also to eliminate side effects.

The term “active agent” as used herein, refers to any agent allowing the treatment, amelioration, and/or prevention of a disease caused by a nidovirus. In addition, the term “active agent”, as used herein, refers to any therapeutic and/or preventive activity an agent may exhibit. The active agent is preferably selected from the group consisting of an antiviral drug, an antibacterial drug, an anti-inflammatory drug, an antifungal drug, an antipyretic agent, and a corticoid.

The birnavirus may be administered to a subject receiving or having received at least one further therapy against a disease caused by a nidovirus. The term “at least one further therapy against a disease caused by a nidovirus”, as used herein, refers to any other therapy than the administration of a birnavirus in order to treat a disease caused by a nidovirus. The at least one further therapy against a disease caused by a nidovirus may be selected from the group consisting of an antiviral drug therapy, an antibacterial drug therapy, an anti-inflammatory drug therapy, an antifungal drug therapy, an antipyretic agent therapy, and a corticoid therapy. Alternatively or additionally, the at least one further therapy against a disease caused by a nidovirus can be ventilation, especially when breath difficulties occur. The ventilation is preferably selected from the group consisting of non-invasive ventilation and invasive ventilation. The non-invasive ventilation is preferably carried out through a face mask, nasal mask, or a helmet, or the invasive ventilation is preferably a mechanic ventilation carried out through a positive pressure ventilator or negative pressure ventilator.

As used herein, the expressions “is for administration” and “is to be administered” have the same meaning as “is prepared to be administered”. In other words, the statement that an active compound “is for administration” has to be understood in that said active compound has been formulated and made up into doses so that said active compound is in a state capable of exerting its therapeutic activity. In the context of the present invention, a birnavirus, a combination comprising at least one birnavirus and at least one further active agent, or a pharmaceutical composition comprising the birnavirus or the combination is prepared to be administered.

The birnavirus, the combination comprising at least one birnavirus and at least one further active agent, or the pharmaceutical composition comprising the birnavirus or the combination is preferably in a form suitable for oral administration, nasal administration, administration by inhalation, intravascular administration, intravenous administration, intramuscular administration, intrathecal administration, subcutaneous administration, or intraperitoneal administration. The birnavirus, the combination comprising at least one birnavirus and at least one further active agent, or the pharmaceutical composition comprising the birnavirus or the combination is more preferably in a form suitable for oral administration, nasal administration, or administration by inhalation.

The terms “therapeutically effective amount” or “therapeutic amount” are intended to mean that amount of a drug or pharmaceutical agent that will elicit the biological or medical response of a tissue, a system, animal or human that is being sought by a researcher, veterinarian, medical doctor or other clinician. The dosage regimen utilizing the birnavirus or the birnavirus in combination with the further active agent as described herein can be selected by the skilled practitioner in accordance with a variety of factors including type, species, age, weight, body mass index, sex and medical condition of the subject; the severity of the condition to be treated; the potency of the compound chosen to be administered; the route of administration; the purpose of the administration; and the renal and hepatic function of the subject. In the context of the present invention, the birnavirus, the combination comprising at least one birnavirus and at least one further active agent, or the pharmaceutical composition comprising the birnavirus or the combination is administered or prepared to be administered in a therapeutically effective/therapeutic amount.

Specifically, the dose at which the birnavirus is to be administered amounts to at least 10⁶ infectious units per day. More specifically, the dose at which the birnavirus is to be administered amounts to between 10⁶ and 10⁹ infectious units per day. The present inventors have determined that the virus is effective at at least 10⁶ infectious units per day and safe up to 10⁹ infectious units per day. In one preferred embodiment, the dose at which the birnavirus is to be administered amounts to ≥10⁶ infectious units and <10⁷ infectious units per day, especially for a maximum of 1 week. In one more preferred embodiment, the dose at which the birnavirus is to be administered amounts to ≥10⁶ infectious units and <10⁷ infectious units once (also dividable into two portions), ≥10⁶ infectious units and <10⁷ infectious units twice within 48 hours, preferably 24 hours, or ≥10⁶ infectious units and <10⁷ infectious units thrice within 72 hours. In one even more preferred embodiment, the dose at which the birnavirus is to be administered amounts to ≥10⁶ infectious units and <10⁷ infectious units once.

The pharmaceutical composition in accordance with the present invention may comprise one or more excipient(s), diluent(s), and/or carrier(s), all of which are preferably pharmaceutically acceptable. The term “pharmaceutically acceptable”, as used herein, means approved by a regulatory agency of the Federal or a state government or listed in the U.S. Pharmacopeia, European Pharmacopeia (Ph. Eur.) or other generally recognized pharmacopeia for use in animals, and more particularly in humans.

The term “excipient”, as used herein, is intended to indicate all substances in a pharmaceutical composition which are not active ingredients such as binders, lubricants, thickeners, surface active agents, preservatives, emulsifiers, buffers, flavoring agents, or colorants.

The term “diluent”, as used herein, relates to a diluting and/or thinning agent. Moreover, the term “diluent” includes a solution, suspension (e.g. liquid or solid suspension) and/or media.

The term “carrier”, as used herein, relates to one or more compatible solid or liquid fillers, which are suitable for an administration, e.g. to a human. The term “carrier” relates to a natural or synthetic organic or inorganic component which is combined with an active component in order to facilitate the application of the active component. Preferably, carrier components are sterile liquids such as water or oils, including those which are derived from mineral oil, animals, or plants, such as peanut oil, soy bean oil, sesame oil, sunflower oil, etc. Salt solutions and aqueous dextrose and glycerin solutions may also be used as aqueous carrier compounds.

Pharmaceutically acceptable carriers or diluents for therapeutic use are well known in the pharmaceutical art, and are described, for example, in Remington's Pharmaceutical Sciences, Mack Publishing Co. (A. R Gennaro edit. 1985). Examples of suitable carriers include, for example, magnesium carbonate, magnesium stearate, talc, lactose, pectin, dextrin, starch, gelatin, tragacanth, methylcellulose, sodium carboxymethylcellulose, a low melting wax, cocoa butter, and the like. Examples of suitable diluents include ethanol, glycerol, and water.

Pharmaceutical carriers, diluents, and/or excipients can be selected with regard to the intended route of administration and standard pharmaceutical practice. The pharmaceutical compositions of the present invention may comprise as, or in addition to, the carrier(s), excipient(s) or diluent(s) any suitable binder(s), lubricant(s), suspending agent(s), coating agent(s), and/or solubilising agent(s). Examples of suitable binders include starch, gelatin, natural sugars such as glucose, lactose, sucrose, trehalose, corn sweeteners, natural and synthetic gums, such as acacia, tragacanth or sodium alginate, carboxymethyl cellulose, and polyethylene glycol. Examples of suitable lubricants include sodium oleate, sodium stearate, magnesium stearate, sodium benzoate, sodium acetate, sodium chloride, and the like. Preservatives, stabilizers, dyes, and even flavoring agents may be provided in the pharmaceutical composition. Examples of preservatives include sodium benzoate, sorbic acid, and esters of p-hydroxybenzoic acid. Antioxidants and suspending agents may be also used.

The term “systemic administration”, a used herein, refers to the administration of the birnavirus, the combination comprising at least one birnavirus and at least one further active agent, or the pharmaceutical composition comprising the birnavirus or the combination such that said birnavirus, combination, or pharmaceutical composition becomes widely distributed in the body of a subject in significant amounts and develops a biological effect. Typical systemic routes of administration include administration by introducing the birnavirus, the combination comprising at least one birnavirus and at least one further active agent, or the pharmaceutical composition comprising the birnavirus or the combination directly into the vascular system, wherein said birnavirus, combination, or pharmaceutical composition enters the vascular system and is carried to one or more desired site(s) of action via the blood. The systemic administration may be by parenteral administration.

The term “parenteral administration”, as used herein, refers to the administration of the birnavirus, the combination comprising at least one birnavirus and at least one further active agent, or the pharmaceutical composition comprising the birnavirus or the combination such that said birnavirus, combination, or pharmaceutical composition does not pass the intestine. The term “parenteral administration” includes intravenous administration, subcutaneous administration, intradermal administration, or intraarterial administration, but is not limited thereto. The birnavirus, the combination comprising at least one birnavirus and at least one further active agent, or a pharmaceutical composition comprising the birnavirus or the combination is preferably administered orally, nasally, or by inhalation.

The term “subject”, as used herein, refers to any individual which may receive a birnavirus, a combination comprising at least one birnavirus and at least one further active agent, or a pharmaceutical composition comprising the birnavirus or the combination of the present invention. The term “subject”, as used herein, refers to any individual that/who may benefit from the treatment with a birnavirus, a combination comprising at least one birnavirus and at least one further active agent, or a pharmaceutical composition comprising the birnavirus or the combination of the present invention. The subject suffers from a disease caused by a nidovirus. The subject is treated for a disease caused by a nidovirus.

The subject may be a vertebrate, e.g. a human being, dog, cat, sheep, goat, cow, horse, camel or pig. It is particularly preferred that the “subject” is a human being.
The terms “subject”, “individual”, or “patient” are used interchangeably herein.

In the context of the present invention, the term “kit of parts (in short: kit)” is understood to be any combination of at least some of the components identified herein, which are combined, coexisting spatially, to a functional unit, and which can contain further components.

### Embodiments of the Invention

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous, unless clearly indicated to the contrary.

The present inventors surprisingly found that viruses of the Birnaviridae family can be used as medicament, specifically as medicament for therapy of diseases caused by a nidovirus such as coronavirus. In particular, the present inventors surprisingly found that viruses of the Birnaviridae family promote activation of the interferon-dependent antiviral gene program and, thus, allow an efficient treatment of a broad range of different nidovirus infections/diseases.

Thus, in a first aspect, the present invention relates to a birnavirus for use in the treatment of a disease caused by a nidovirus.

In one preferred embodiment, the birnavirus is selected from the group consisting of an avibirnavirus, an aquabirnavirus, a blosnavirus, a dronavirus, an entomobirnavirus, a ronavirus, and a telnavirus. In one more preferred embodiment, the avibirnavirus is an Infectious Bursal Disease Virus (IBDV). In one even more preferred embodiment, the IBDV is an IBDV of strain 903/78. In one still even more preferred embodiment, the IBDV of strain 903/78 comprises a nucleotide sequence according to SEQ ID NO: 1, a fragment thereof or a sequence having at least 80% sequence identity thereto, and/or a nucleotide sequence according to SEQ ID NO: 2, a fragment thereof or a sequence having at least 80% sequence identity thereto.

In particular, the nucleotide sequence can be selected from the group consisting of
(i) a nucleotide sequence according to SEQ ID NO: 1 or SEQ ID NO: 2,
(ii) a nucleotide sequence that is a fragment of the nucleotide sequence according to (i), and
(iii) a nucleotide sequence that has at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

Preferably, the birnavirus is a live and/or attenuated birnavirus. The birnavirus may be a naturally occurring or non-naturally occurring (live and/or attenuated) birnavirus. The non-naturally occurring birnavirus is preferably a recombinant birnavirus. The naturally or non-naturally occurring birnavirus may be rescued from a bacterial plasmid designed for expression of birnavirus RNAs. The non-naturally occurring (recombinant) birnavirus is preferably a mutated birnavirus or a chemically treated birnavirus (e.g. treated with a protease).

In one preferred embodiment, the nidovirus is a virus of the family of Coronaviridae, Arteriviridae, Roniviridae, or Mesoniviridae. In one more preferred embodiment, the nidovirus is a virus of the family of Coronaviridae or Arteriviridae. Within the Coronaviridae, one subfamily is termed Orthocoronavirinae and the four genera therein are alpha-coronavirus, beta-coronavirus, delta-coronavirus and gamma-coronavirus. The alpha- and beta-coronaviruses appear to have evolved in bat reservoirs and the delta- and gamma-coronaviruses appear to have evolved in avian reservoirs. The coronavirus may be an alpha-coronavirus or a beta-coronavirus. Examples of alpha-coronaviruses are HCoV-229E and CoV-NL63, and examples of beta-coronaviruses are SARS-CoV, SARS-CoV-2, MERS-CoV, HCoV-HKU1, and HCoV-OC43. In one even more preferred embodiment, the virus of the family of Coronaviridae is selected from the group consisting of MERS-CoV, SARS-CoV, SARS-CoV-2, a coronavirus that gains entry by the ACE2 receptor, and an Infectious Bronchitis Virus (IBV). In one alternative even more preferred embodiment, the virus of the family of Arteriviridae is selected from the group consisting of a virus that causes porcine reproductive and respiratory syndrome (PRRS) and a virus that causes equine viral arteritits (EVA).

In one particularly preferred embodiment, the birnavirus is for use in the treatment of a disease caused by a nidovirus, wherein the birnavirus is an Infectious Bursal Disease Virus (IBDV) and wherein the disease caused by a nidovirus is a severe acute respiratory syndrome (SARS) such as COVID-19. In one particularly more preferred embodiment, the birnavirus is for use in the treatment of a disease caused by a nidovirus, wherein the birnavirus is an Infectious Bursal Disease Virus (IBDV) and wherein the disease caused by a nidovirus is a mild or moderate form of a severe acute respiratory syndrome (SARS) such as COVID-19. In other words: In one particularly more preferred embodiment, the birnavirus is for use in the treatment of a disease caused by a nidovirus, wherein the birnavirus is an Infectious Bursal Disease Virus (IBDV) and wherein the disease caused by a nidovirus is a severe acute respiratory syndrome (SARS) such as COVID-19 not associated with an acute respiratory distress syndrome.

By using the clinically proven attenuated live avian virus IBDV R903/78 for the treatment of a disease caused by a nidovirus, in particular SARS-CoV-2, the present inventors surprisingly found that a dose of at least 10⁶ infectious units per day is effective in the treatment of a disease caused by a nidovirus, in particular SARS-CoV-2, in humans and that a dose of 10⁹ infectious units per day is still safe for humans. Thus, the dose at which the birnavirus is to be administered specifically amounts to at least 10⁶ infectious units per day. More specifically, the dose at which the birnavirus is to be administered amounts to between 10⁶ and 10⁹ infectious units per day. It should be noted that the above doses are preferably to be administered for no more than/for a maximum of 1 week, e.g. for 1, 2, 3, 4, 5, 6, or 7 day(s).

In one preferred embodiment, the dose at which the birnavirus is to be administered amounts to ≥10⁶ infectious units and <10⁷ infectious units per day. In one more preferred embodiment, the dose at which the birnavirus is to be administered amounts to ≥10⁶ infectious units and <10⁷ infectious units per day for no more than/for a maximum of 1 week, e.g. for 1, 2, 3, 4, 5, 6, or 7 day(s). In one even more preferred embodiment, the dose at which the birnavirus is to be administered amounts to ≥10⁶ infectious units and <10⁷ infectious units once (also dividable into two portions), ≥10⁶ infectious units and <10⁷ infectious units twice within 48 hours, preferably 24 hours, or ≥10⁶ infectious units and <10⁷ infectious units thrice within 72 hours. In one still even more preferred embodiment, the dose at which the birnavirus is to be administered amounts to ≥10⁶ infectious units and <10⁷ infectious units once. Preferably, the above described doses are to be administered within 1 week after the first sign(s)/symptom(s) of a disease caused by a nidovirus, e.g. SARS-CoV-2. For example, the above described doses are to be administered after 1, 2, 3, 4, 5, 6, or 7 day(s) after the first sign(s)/symptom(s) of a disease caused by a nidovirus, e.g. SARS-CoV-2. More preferably, the above described doses are to be administered immediately after the first sign(s)/symptom(s) of a disease caused by a nidovirus, e.g. SARS-CoV-2.

Thus, in one particularly preferred embodiment, the dose at which the birnavirus is to be administered amounts to ≥10⁶ infectious units and <10⁷ infectious units per day, wherein the dose is to be administered within 1 week after the first sign(s)/symptom(s) of a disease caused by a nidovirus such as SARS-CoV-2, e.g. after 1, 2, 3, 4, 5, 6, or 7 day(s). In one particularly more preferred embodiment, the dose at which the birnavirus is to be administered amounts to ≥10⁶ infectious units and <10⁷ infectious units per day for no more than/for a maximum of 1 week, wherein the dose is to be administered within 1 week after the first sign(s)/symptom(s) of a disease caused by a nidovirus such as SARS-CoV-2, e.g. after 1, 2, 3, 4, 5, 6, or 7 day(s). In one particularly even more preferred embodiment, the dose at which the birnavirus is to be administered amounts to ≥10⁶ infectious units and <10⁷ infectious units once (also dividable into two portions), ≥10⁶ infectious units and <10⁷ infectious units twice within 48 hours, preferably 24 hours, or ≥10⁶ infectious units and <10⁷ infectious units thrice within 72 hours, wherein the dose is to be administered within 1 week after the first sign(s)/symptom(s) of a disease caused by a nidovirus such as SARS-CoV-2, e.g. after 1, 2, 3, 4, 5, 6, or 7 day(s). In one particularly still even more preferred embodiment, the dose at which the birnavirus is to be administered amounts to ≥10⁶ infectious units and <10⁷ infectious units once, wherein the dose is to be administered within 1 week after the first sign(s)/symptom(s) of a disease caused by a nidovirus such as SARS-CoV-2, e.g. after 1, 2, 3, 4, 5, 6, or 7 day(s). Preferably, the above described birnavirus is an Infectious Bursal Disease Virus (IBDV) and the disease caused by a nidovirus is a severe acute respiratory syndrome (SARS) such as COVID-19. More preferably, the above described birnavirus is an Infectious Bursal Disease Virus (IBDV) and the disease caused by a nidovirus is a mild or moderate form of a severe acute respiratory syndrome (SARS) such as COVID-19. In other words: More preferably, the above described birnavirus is an Infectious Bursal Disease Virus (IBDV) and the disease caused by a nidovirus is a severe acute respiratory syndrome (SARS) such as COVID-19 not associated with an acute respiratory distress syndrome.

In addition to the first appearance of nidovirus disease symptoms as a signal to start therapy, a positive nidovirus disease test result can also mark the start of therapy.

In one preferred embodiment, the birnavirus is for administration to a subject receiving or having received at least one further therapy against a disease caused by a nidovirus such as SARS-CoV-2. In this regard, the administration of the birnavirus is considered to be a first therapy against a disease caused by a nidovirus and the further therapy against a disease caused by a nidovirus is considered to be a second therapy against a disease caused by a nidovirus.

In one preferred embodiment, the at least one further therapy against a disease caused by a nidovirus is selected from the group consisting of an antiviral drug therapy, an antibacterial drug therapy, an anti-inflammatory drug therapy, antifungal drug therapy, an antipyretic agent therapy, and a corticoid therapy.

In one more preferred embodiment,
(i) the antiviral drug is selected from the group consisting of chloroquine, remdesivir, darunavir, favipiravir, lopinavir, and ritonavir, or is a combination thereof,
(ii) the antibacterial drug is selected from the group consisting of ceftriaxone, amoxicillin/clavulanic acid, piperacillin/tazobactam, and azithromycin, or is a combination thereof,
(iii) the anti-inflammatory drug is selected from the group consisting of ibuprofen and metamizole,
(iv) the antifungal drug is selected from the group consisting of voriconazole and isavuconazole,
(v) the antipyretic agent is paracetamol, and/or
(vi) the corticoid is dexamethasone.

However, also (additionally or alternatively) another treatment than a drug therapy is possible. Another form of therapeutic treatment is preferably ventilation. More preferably, the ventilation is selected from the group consisting of non-invasive ventilation and invasive ventilation. Even more preferably, the non-invasive ventilation is carried out through a face mask, nasal mask, or a helmet, or the invasive ventilation is a mechanic ventilation, e.g. carried out through a positive pressure ventilator or negative pressure ventilator.

The birnavirus is for use in the treatment of a disease caused by a nidovirus in a subject. The subject may be a mammal selected from the group consisting of a human being, dog, cat, sheep, goat, cow, horse, camel and pig. It is particularly preferred that the mammal is a human being.

In one most preferred embodiment the present invention relates to an Infectious Bursal Disease Virus (IBDV) for use in the treatment of a mild or moderate form of COVID-19, in particular COVID-19 not associated with an acute respiratory distress syndrome. In this most preferred embodiment, the birnavirus is to be administered in an amount of between 10⁶ and 10⁹ infectious units per day, in particular ≥10⁶ infectious units and <10⁷ infectious units per day, for no more than/for a maximum of 1 week, e.g. for 1, 2, 3, 4, 5, 6, or 7 day(s), and within 1 week after the first sign(s)/symptom(s) of COVID-19, e.g. after 1, 2, 3, 4, 5, 6, or 7 day(s). In addition to the first appearance of COVID-19 symptoms as a signal to start therapy, a positive COVID-19 test result can also mark the start of therapy. In one particularly most preferred embodiment, the Infectious Bursal Disease Virus (IBDV) is to be administered, at the dosage described above, orally, nasally, or by inhalation.

The first aspect of the present invention can alternatively be worded as follows: A method for treating a disease caused by a nidovirus comprising the step of: administering a birnavirus to a subject (in need thereof), thereby treating the disease caused by a nidovirus in the subject.

It is preferred that the birnavirus is administered in a therapeutically effective amount. It is (alternatively or additionally) further preferred that the subject is treated with the birnavirus prior to, during and/or after the subject was subjected to at least one further therapy against a disease caused by a nidovirus. It is more preferred that the at least one further therapy against a disease caused by a nidovirus is selected from the group consisting of an antiviral drug therapy, an antibacterial drug therapy, an anti-inflammatory drug therapy, antifungal drug therapy, an antipyretic agent therapy, and a corticoid therapy.

With regard to the preferred embodiments, reference is made to the above explanations.

The first aspect of the present invention can further alternatively be worded as follows: Use of a birnavirus for the preparation of a medicament for the treatment of a disease caused by a nidovirus.

With regard to the preferred embodiments, reference is made to the above explanations.

In a second aspect, the present invention relates to a combination comprising at least one birnavirus and at least one further active agent for use in the treatment of a disease caused by a nidovirus. Thus, the at least one birnavirus and the at least one further active agent are used in a combination therapy.

The at least one birnavirus and the at least one further active agent may be present in the combination individually or together. For example, the at least one birnavirus may be comprised in a (first) composition and the at least one further active agent may be comprised in another/different (second) composition. Alternatively, the at least one birnavirus and the at least one further active agent may be comprised in a single composition.

In one preferred embodiment, the birnavirus is selected from the group consisting of an avibirnavirus, an aquabirnavirus, a blosnavirus, a dronavirus, an entomobirnavirus, a ronavirus, and a telnavirus. In one more preferred embodiment, the avibirnavirus is an Infectious Bursal Disease Virus (IBDV). In one even more preferred embodiment, the IBDV is an IBDV of strain 903/78. In one still even more preferred embodiment, the IBDV of strain 903/78 comprises a nucleotide sequence according to SEQ ID NO: 1 (segment A), a fragment thereof or a sequence having at least 80% sequence identity thereto, and/or a nucleotide sequence according to SEQ ID NO: 2 (segment B), a fragment thereof or a sequence having at least 80% sequence identity thereto.

In particular, the nucleotide sequence can be selected from the group consisting of
(i) a nucleotide sequence according to SEQ ID NO: 1 or SEQ ID NO: 2,
(ii) a nucleotide sequence that is a fragment of the nucleotide sequence according to (i), and
(iii) a nucleotide sequence that has at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

Preferably, the birnavirus is a live and/or attenuated birnavirus. The birnavirus may be a naturally occurring or non-naturally occurring (live and/or attenuated) birnavirus. The non-naturally occurring birnavirus is preferably a recombinant birnavirus. The non-naturally occurring birnavirus is preferably a mutated birnavirus or a chemically treated birnavirus (e.g. treated with a protease).

In one preferred embodiment, the nidovirus is a virus of the family of Coronaviridae, Arteriviridae, Roniviridae, or Mesoniviridae. In one more preferred embodiment, the nidovirus is a virus of the family of Coronaviridae or Arteriviridae. Within the Coronaviridae, one subfamily is termed Orthocoronavirinae and the four genera therein are alpha-coronavirus, beta-coronavirus, delta-coronavirus and gamma-coronavirus. The alpha- and beta-coronaviruses appear to have evolved in bat reservoirs and the delta- and gamma-coronaviruses appear to have evolved in avian reservoirs. The coronavirus may be an alpha-coronavirus or a beta-coronavirus. Examples of alpha-coronaviruses are HCoV-229E and CoV-NL63, and examples of beta-coronaviruses are SARS-CoV, SARS-CoV-2, MERS-CoV, HCoV-HKU1, and HCoV-OC43. In one even more preferred embodiment, the virus of the family of Coronaviridae is selected from the group consisting of MERS-CoV, SARS-CoV, SARS-CoV-2, a coronavirus that gains entry by the ACE2 receptor, and an Infectious Bronchitis Virus (IBV). In one alternative even more preferred embodiment, the virus of the family of Arteriviridae is selected from the group consisting of a virus that causes porcine reproductive and respiratory syndrome (PRRS) and a virus that causes equine viral arteritits (EVA).

In one particularly preferred embodiment, the birnavirus is for use in the treatment of a disease caused by a nidovirus, wherein the birnavirus is an Infectious Bursal Disease Virus (IBDV) and wherein the disease caused by a nidovirus is a severe acute respiratory syndrome (SARS) such as COVID-19. In one particularly more preferred embodiment, the birnavirus is for use in the treatment of a disease caused by a nidovirus, wherein the birnavirus is an Infectious Bursal Disease Virus (IBDV) and wherein the disease caused by a nidovirus is a mild or moderate form of a severe acute respiratory syndrome (SARS) such as COVID-19. In other words: In one particularly more preferred embodiment, the birnavirus is for use in the treatment of a disease caused by a nidovirus, wherein the birnavirus is an Infectious Bursal Disease Virus (IBDV) and wherein the disease caused by a nidovirus is a severe acute respiratory syndrome (SARS) such as COVID-19 not associated with an acute respiratory distress syndrome.

By using the clinically proven attenuated live avian virus IBDV R903/78 for the treatment of a disease caused by a nidovirus, in particular SARS-CoV-2, the present inventors surprisingly found that a dose of at least 10⁶ infectious units per day is effective in the treatment of a disease caused by a nidovirus, in particular SARS-CoV-2, in humans and that a dose of 10⁹ infectious units per day is still safe for humans. Thus, the dose at which the birnavirus is to be administered specifically amounts to at least 10⁶ infectious units per day. More specifically, the dose at which the birnavirus is to be administered amounts to between 10⁶ and 10⁹ infectious units per day. It should be noted that the above doses are preferably to be administered for no more than/for a maximum of 1 week, e.g. for 1, 2, 3, 4, 5, 6, or 7 day(s).

In one preferred embodiment, the dose at which the birnavirus is to be administered amounts to ≥10⁶ infectious units and <10⁷ infectious units per day. In one more preferred embodiment, the dose at which the birnavirus is to be administered amounts to ≥10⁶ infectious units and <10⁷ infectious units per day for no more than/for a maximum of 1 week, e.g. for 1, 2, 3, 4, 5, 6, or 7 day(s). In one even more preferred embodiment, the dose at which the birnavirus is to be administered amounts to ≥10⁶ infectious units and <10⁷ infectious units once (also dividable into two portions), ≥10⁶ infectious units and <10⁷ infectious units twice within 48 hours, preferably 24 hours, or ≥10⁶ infectious units and <10⁷ infectious units thrice within 72 hours. In one still even more preferred embodiment, the dose at which the birnavirus is to be administered amounts to ≥10⁶ infectious units and <10⁷ infectious units once. Preferably, the above described doses are to be administered within 1 week after the first sign(s)/symptom(s) of a disease caused by a nidovirus, e.g. SARS-CoV-2. For example, the above described doses are to be administered after 1, 2, 3, 4, 5, 6, or 7 day(s) after the first sign(s)/symptom(s) of a disease caused by a nidovirus, e.g. SARS-CoV-2. More preferably, the above described doses are to be administered immediately after the first sign(s)/symptom(s) of a disease caused by a nidovirus, e.g. SARS-CoV-2.

Thus, in one particularly preferred embodiment, the dose at which the birnavirus is to be administered amounts to ≥10⁶ infectious units and <10⁷ infectious units per day, wherein the dose is to be administered within 1 week after the first sign(s)/symptom(s) of a disease caused by a nidovirus such as SARS-CoV-2, e.g. after 1, 2, 3, 4, 5, 6, or 7 day(s). In one particularly more preferred embodiment, the dose at which the birnavirus is to be administered amounts to ≥10⁶ infectious units and <10⁷ infectious units per day for no more than/for a maximum of 1 week, wherein the dose is to be administered within 1 week after the first sign(s)/symptom(s) of a disease caused by a nidovirus such as SARS-CoV-2, e.g. after 1, 2, 3, 4, 5, 6, or 7 day(s). In one particularly even more preferred embodiment, the dose at which the birnavirus is to be administered amounts to ≥10⁶ infectious units and <10⁷ infectious units once (also dividable into two portions), ≥10⁶ infectious units and <10⁷ infectious units twice within 48 hours, preferably 24 hours, or ≥10⁶ infectious units and <10⁷ infectious units thrice within 72 hours, wherein the dose is to be administered within 1 week after the first sign(s)/symptom(s) of a disease caused by a nidovirus such as SARS-CoV-2, e.g. after 1, 2, 3, 4, 5, 6, or 7 day(s). In one particularly still even more preferred embodiment, the dose at which the birnavirus is to be administered amounts to ≥10⁶ infectious units and <10⁷ infectious units once, wherein the dose is to be administered within 1 week after the first sign(s)/symptom(s) of a disease caused by a nidovirus such as SARS-CoV-2, e.g. after 1, 2, 3, 4, 5, 6, or 7 day(s).

Preferably, the above described birnavirus is an Infectious Bursal Disease Virus (IBDV) and the disease caused by a nidovirus is a severe acute respiratory syndrome (SARS) such as COVID-19. More preferably, the above described birnavirus is an Infectious Bursal Disease Virus (IBDV) and the disease caused by a nidovirus is a mild or moderate form of a severe acute respiratory syndrome (SARS) such as COVID-19. In other words: More preferably, the above described birnavirus is an Infectious Bursal Disease Virus (IBDV) and the disease caused by a nidovirus is a severe acute respiratory syndrome (SARS) such as COVID-19 not associated with an acute respiratory distress syndrome.

In one preferred embodiment, the combination comprising at least one birnavirus and at least one further active agent is for use in the treatment of a disease caused by a nidovirus such as SARS-CoV-2, wherein the at least one further active agent is selected from the group consisting of an antiviral drug, an antibacterial drug, an anti-inflammatory drug, antifungal drug, an antipyretic agent, and a corticoid.

In one more preferred embodiment,
(i) the antiviral drug is selected from the group consisting of chloroquine, remdesivir, darunavir, favipiravir, lopinavir, and ritonavir, or is a combination thereof,
(ii) the antibacterial drug is selected from the group consisting of ceftriaxone, amoxicillin/clavulanic acid, piperacillin/tazobactam, and azithromycin, or is a combination thereof,
(iii) the anti-inflammatory drug is selected from the group consisting of ibuprofen and metamizole,
(iv) the antifungal drug is selected from the group consisting of voriconazole and isavuconazole,
(v) the antipyretic agent is paracetamol, and/or
(vi) the corticoid is dexamethasone.

The at least one birnavirus and the at least one further active agent can be administered concurrently or consecutively. In those embodiments, in which the at least one birnavirus and the at least one further active agent are to be administered concurrently, said at least one birnavirus and said at least one further agent are preferably comprised in the combination together, e.g. in one single composition. In those embodiments, in which the at least one birnavirus and the at least one further active agent are to be administered consecutively, said at least one birnavirus and said at least one further agent are preferably comprised in the combination individually, e.g. the at least one birnavirus may be comprised in a (first) composition and the at least one further active agent may be comprised in another/different (second) composition.

It is particularly preferred that the combination comprises different birnaviruses (i.e. a mixture of birnaviruses). The different birnaviruses are preferably birnaviruses of different types or from different strains.

The combination is for use in the treatment of a disease caused by a nidovirus in a subject. The subject suffers from a disease caused by a nidovirus. The subject may be a mammal selected from the group consisting of a human being, dog, cat, sheep, goat, cow, horse, camel and pig. It is particularly preferred that the mammal is a human being.

In one most preferred embodiment the present invention relates to a combination comprising at least one Infectious Bursal Disease Virus (IBDV) and at least one further active agent for use in the treatment of a mild or moderate form of COVID-19, in particular COVID-19 not associated with an acute respiratory distress syndrome. In this most preferred embodiment, the birnavirus is to be administered in an amount of between 10⁶ and 10⁹ infectious units per day, in particular ≥10⁶ infectious units and <10⁷ infectious units per day, for no more than/for a maximum of 1 week, e.g. for 1, 2, 3, 4, 5, 6, or 7 day(s), and within 1 week after the first sign(s)/symptom(s) of COVID-19, e.g. after 1, 2, 3, 4, 5, 6, or 7 day(s). In addition to the first appearance of COVID-19 symptoms as a signal to start therapy, a positive COVID-19 test result can also mark the start of therapy. In one particularly most preferred embodiment, the Infectious Bursal Disease Virus (IBDV) is to be administered, at the dosage described above, orally, nasally, or by inhalation.

The second aspect of the present invention can alternatively be worded as follows: A method for treating a disease caused by a nidovirus comprising the step of: administering a combination comprising at least one birnavirus and at least one further active agent to a subject (in need thereof), thereby treating the disease caused by a nidovirus in the subject.

It is preferred that the component(s) of the combination, i.e. the at least birnavirus and/or the at least one active agent, is (are) administered in a therapeutically effective amount. With regard to the preferred embodiments, reference is made to the above explanations.

The second aspect of the present invention can further alternatively be worded as follows: Use of a combination comprising at least one birnavirus and at least one further active agent for the preparation of a medicament for the treatment of a disease caused by a nidovirus. With regard to the preferred embodiments, reference is made to the above explanations.

In a third aspect, the present invention relates to a pharmaceutical composition comprising a birnavirus as defined in the first aspect or a combination as defined in the second aspect for use in the treatment of a disease caused by a nidovirus.

In one preferred embodiment, the nidovirus is a virus of the family of Coronaviridae, Arteriviridae, Roniviridae, or Mesoniviridae. In one more preferred embodiment, the nidovirus is a virus of the family of Coronaviridae or Arteriviridae. Within the Coronaviridae, one subfamily is termed Orthocoronavirinae and the four genera therein are alpha-coronavirus, beta-coronavirus, delta-coronavirus and gamma-coronavirus. The alpha- and beta-coronaviruses appear to have evolved in bat reservoirs and the delta- and gamma-coronaviruses appear to have evolved in avian reservoirs. The coronavirus may be an alpha-coronavirus or a beta-coronavirus. Examples of alpha-coronaviruses are HCoV-229E and CoV-NL63, and examples of beta-coronaviruses are SARS-CoV, SARS-CoV-2, MERS-CoV, HCoV-HKU1, and HCoV-OC43. In one even more preferred embodiment, the virus of the family of Coronaviridae is selected from the group consisting of MERS-CoV, SARS-CoV, SARS-CoV-2, a coronavirus that gains entry by the ACE2 receptor, and an Infectious Bronchitis Virus (IBV). In one alternative even more preferred embodiment, the virus of the family of Arteriviridae is selected from the group consisting of a virus that causes porcine reproductive and respiratory syndrome (PRRS) and a virus that causes equine viral arteritits (EVA).

In one preferred embodiment, the pharmaceutical composition comprises one or more pharmaceutical acceptable excipient(s), diluent(s), and/or carrier(s).

The pharmaceutical composition can be administered systemically, e.g. parenterally. In one preferred embodiment, the pharmaceutical composition is in a form suitable for oral administration, nasal administration, or administration by inhalation. The pharmaceutical composition can also be administered intravascular, intravenous, intramuscular, intrathecal, subcutaneous, or intraperitoneal.

The pharmaceutical composition can be administered in a single dose or in more than one dose. It is preferred that the pharmaceutical composition is to be administered in a therapeutically effective amount.

By using the clinically proven attenuated live avian virus IBDV R903/78 for the treatment of a disease caused by a nidovirus, in particular SARS-CoV-2, the present inventors surprisingly found that a dose of at least 10⁶ infectious units per day is effective in the treatment of a disease caused by a nidovirus, in particular SARS-CoV-2, in humans and that a dose of 10⁹ infectious units per day is still safe for humans. Thus, the dose at which the birnavirus is to be administered specifically amounts to at least 10⁶ infectious units per day. More specifically, the dose at which the birnavirus is to be administered amounts to between 10⁶ and 10⁹ infectious units per day. It should be noted that the above doses are preferably to be administered for no more than/for a maximum of 1 week, e.g. for 1, 2, 3, 4, 5, 6, or 7 day(s).

In one preferred embodiment, the dose at which the birnavirus is to be administered amounts to ≥10⁶ infectious units and <10⁷ infectious units per day. In one more preferred embodiment, the dose at which the birnavirus is to be administered amounts to ≥10⁶ infectious units and <10⁷ infectious units per day for no more than/for a maximum of 1 week, e.g. for 1, 2, 3, 4, 5, 6, or 7 day(s). In one even more preferred embodiment, the dose at which the birnavirus is to be administered amounts to ≥10⁶ infectious units and <10⁷ infectious units once (also dividable into two portions), ≥10⁶ infectious units and <10⁷ infectious units twice within 48 hours, preferably 24 hours, or ≥10⁶ infectious units and <10⁷ infectious units thrice within 72 hours. In one still even more preferred embodiment, the dose at which the birnavirus is to be administered amounts to ≥10⁶ infectious units and <10⁷ infectious units once. Preferably, the above described doses are to be administered within 1 week after the first sign(s)/symptom(s) of a disease caused by a nidovirus, e.g. SARS-CoV-2. For example, the above described doses are to be administered after 1, 2, 3, 4, 5, 6, or 7 day(s) after the first sign(s)/symptom(s) of a disease caused by a nidovirus, e.g. SARS-CoV-2. More preferably, the above described doses are to be administered immediately after the first sign(s)/symptom(s) of a disease caused by a nidovirus, e.g. SARS-CoV-2.

Thus, in one particularly preferred embodiment, the dose at which the birnavirus is to be administered amounts to ≥10⁶ infectious units and <10⁷ infectious units per day, wherein the dose is to be administered within 1 week after the first sign(s)/symptom(s) of a disease caused by a nidovirus such as SARS-CoV-2, e.g. after 1, 2, 3, 4, 5, 6, or 7 day(s). In one particularly more preferred embodiment, the dose at which the birnavirus is to be administered amounts to ≥10⁶ infectious units and <10⁷ infectious units per day for no more than/for a maximum of 1 week, wherein the dose is to be administered within 1 week after the first sign(s)/symptom(s) of a disease caused by a nidovirus such as SARS-CoV-2, e.g. after 1, 2, 3, 4, 5, 6, or 7 day(s). In one particularly even more preferred embodiment, the dose at which the birnavirus is to be administered amounts to ≥10⁶ infectious units and <10⁷ infectious units once (also dividable into two portions), ≥10⁶ infectious units and <10⁷ infectious units twice within 48 hours, preferably 24 hours, or ≥10⁶ infectious units and <10⁷ infectious units thrice within 72 hours, wherein the dose is to be administered within 1 week after the first sign(s)/symptom(s) of a disease caused by a nidovirus such as SARS-CoV-2, e.g. after 1, 2, 3, 4, 5, 6, or 7 day(s). In one particularly still even more preferred embodiment, the dose at which the birnavirus is to be administered amounts to ≥10⁶ infectious units and <10⁷ infectious units once, wherein the dose is to be administered within 1 week after the first sign(s)/symptom(s) of a disease caused by a nidovirus such as SARS-CoV-2, e.g. after 1, 2, 3, 4, 5, 6, or 7 day(s). Preferably, the above described birnavirus is an Infectious Bursal Disease Virus (IBDV) and the disease caused by a nidovirus is a severe acute respiratory syndrome (SARS) such as COVID-19. More preferably, the above described birnavirus is an Infectious Bursal Disease Virus (IBDV) and the disease caused by a nidovirus is a mild or moderate form of a severe acute respiratory syndrome (SARS) such as COVID-19. In other words: More preferably, the above described birnavirus is an Infectious Bursal Disease Virus (IBDV) and the disease caused by a nidovirus is a severe acute respiratory syndrome (SARS) such as COVID-19 not associated with an acute respiratory distress syndrome.

The pharmaceutical composition is for use in the treatment of a disease caused by a nidovirus in a subject. The subject suffers from a disease caused by a nidovirus. The subject may be a mammal selected from the group consisting of a human being, dog, cat, sheep, goat, cow, horse, camel and pig. It is particularly preferred that the mammal is a human being.

The third aspect of the present invention can alternatively be worded as follows: A method for treating a disease caused by a nidovirus comprising the step of: administering a pharmaceutical composition comprising a birnavirus as defined in the first aspect or a combination as defined in the second aspect to a subject (in need thereof), thereby treating the disease caused by a nidovirus in the subject.

With regard to the preferred embodiments, reference is made to the above explanations.

The third aspect of the present invention can further alternatively be worded as follows: Use of a pharmaceutical composition comprising a birnavirus as defined in the first aspect or a combination as defined in the second aspect for the preparation of a medicament for the treatment of a disease caused by a nidovirus.

With regard to the preferred embodiments, reference is made to the above explanations.

In a fourth aspect, the present invention relates to a reservoir comprising a birnavirus as defined in the first aspect, a combination as defined in the second aspect, or a pharmaceutical composition as defined in the third aspect, wherein the reservoir is designed to be inserted into an oral or a nasal applicator.

The reservoir may comprise or consist of one or more chambers for receiving a birnavirus as defined in the first aspect, a combination as defined in the second aspect, or a pharmaceutical composition as defined in the third aspect.

In those embodiments, in which only the birnavirus, the combination comprising the at least one birnavirus and the at least one further active agent together, or the pharmaceutical composition comprising the birnavirus or the at least one birnavirus and the at least one further active agent is to be administered, the reservoir comprises only one chamber. In those embodiments, in which the combination comprising the at least one birnavirus and the at least one further active agent individually is to be administered, the reservoir comprises two chambers, i.e. one chamber for the at least one birnavirus and one chamber for the at least one further active agent.

In a fifth aspect, the present invention relates to an oral or a nasal applicator comprising
(i) a reservoir comprising a birnavirus as defined in the first aspect, a combination as defined in the second aspect, or a pharmaceutical composition as defined in the third aspect, and
(ii) a mouth piece or nose piece, wherein the mouth piece or the nose piece is connected with/attached to the reservoir.

The oral or a nasal applicator allows the orally or nasally application of a birnavirus as defined in the first aspect, a combination as defined in the second aspect, or a pharmaceutical composition as defined in the third aspect.

The reservoir may comprise or consist of one or more chambers for receiving a birnavirus as defined in the first aspect, a combination as defined in the second aspect, or a pharmaceutical composition as defined in the third aspect.

In those embodiments, in which only the birnavirus, the combination comprising the at least one birnavirus and the at least one further active agent together, or the pharmaceutical composition comprising the birnavirus or the at least one birnavirus and the at least one further active agent is to be administered, the reservoir comprises only one chamber. In those embodiments, in which the combination comprising the at least one birnavirus and the at least one further active agent individually is to be administered, the reservoir comprises two chambers, i.e. one chamber for the at least one birnavirus and one chamber for the at least one further active agent.

In one preferred embodiment, the mouth piece or nose piece is designed so as to allow complete insertion into the mouth or nose.

In one further preferred embodiment, the oral or nasal applicator also comprises a discharge dosing unit for dosed release of a birnavirus as defined in the first aspect, a combination as defined in the second aspect, or a pharmaceutical composition as defined in the third aspect from the reservoir via the mouth or nose piece.

Preferably, the oral or nasal applicator is configured to administer a dose of the birnavirus which amounts to at least 10⁶ infectious units. More preferably, the oral or nasal applicator is configured to administer a dose of the birnavirus which amounts to between 10⁶ and 10⁹ infectious units. Even more preferably, the oral or nasal applicator is configured to administer a dose of the birnavirus which amounts to ≥10⁶ infectious units and <10⁷ infectious units. The birnavirus is particularly IBDV.

The oral or a nasal applicator is preferably used for the treatment of a disease caused by a nidovirus in a subject. The subject suffers from a disease caused by a nidovirus. The subject may be a mammal selected from the group consisting of a human being, dog, cat, sheep, goat, cow, horse, camel and pig. It is particularly preferred that the mammal is a human being. As to the specific diseases caused by a nidovirus, it is referred to the first aspect of the present invention.

In a sixth aspect, the present invention relates to a kit comprising
(i) a packaging material, and
(ii) a birnavirus as defined in the first aspect, a combination as defined in the second aspect, a pharmaceutical composition as defined in the third aspect, a reservoir of the fourth aspect, or an oral or nasal applicator of the fifth aspect.

The kit is preferably used for the treatment of a disease caused by a nidovirus in a subject. The subject suffers from a disease caused by a nidovirus. The subject may be a mammal selected from the group consisting of a human being, dog, cat, sheep, goat, cow, horse, camel and pig. It is particularly preferred that the mammal is a human being. As to the specific diseases caused by a nidovirus, it is referred to the first aspect of the present invention.

In one preferred embodiment, the kit further comprises
(iii) a label or packaging insert contained within the packaging material indicating that subjects receiving the birnavirus, the combination or the pharmaceutical composition can be treated for a disease caused by a nidovirus.

As to the specific diseases caused by a nidovirus, it is referred to the first aspect of the present invention.

Specifically, the label or packaging insert further comprises the information that the dose at which the birnavirus is to be administered amounts to at least 10⁶ infectious units per day. More specifically, the label or packaging insert further comprises the information that the dose at which the birnavirus is to be administered amounts to between 10⁶ and 10⁹ infectious units per day. It should be noted that the above doses are preferably to be administered for no more than/for a maximum of 1 week, e.g. for 1, 2, 3, 4, 5, 6, or 7 day(s).

In one preferred embodiment, the dose at which the birnavirus is to be administered amounts to ≥10⁶ infectious units and <10⁷ infectious units per day. In one more preferred embodiment, the dose at which the birnavirus is to be administered amounts to ≥10⁶ infectious units and <7 infectious units per day for no more than/for a maximum of 1 week, e.g. for 1, 2, 3, 4, 5, 6, or 7 day(s). In one even more preferred embodiment, the dose at which the birnavirus is to be administered amounts to ≥10⁶ infectious units and <10⁷ infectious units once (also dividable into two portions), ≥10⁶ infectious units and <10⁷ infectious units twice within 48 hours, preferably 24 hours, or ≥10⁶ infectious units and <10⁷ infectious units thrice within 72 hours. In one still even more preferred embodiment, the dose at which the birnavirus is to be administered amounts to ≥10⁶ infectious units and <10⁷ infectious units once. Preferably, the above described doses are to be administered within 1 week after the first sign(s)/symptom(s) of a disease caused by a nidovirus, e.g. SARS-CoV-2. For example, the above described doses are to be administered after 1, 2, 3, 4, 5, 6, or 7 day(s) after the first sign(s)/symptom(s) of a disease caused by a nidovirus, e.g. SARS-CoV-2. More preferably, the above described doses are to be administered immediately after the first sign(s)/symptom(s) of a disease caused by a nidovirus, e.g. SARS-CoV-2.

Thus, in one particularly preferred embodiment, the dose at which the birnavirus is to be administered amounts to ≥10⁶ infectious units and <10⁷ infectious units per day, wherein the dose is to be administered within 1 week after the first sign(s)/symptom(s) of a disease caused by a nidovirus such as SARS-CoV-2, e.g. after 1, 2, 3, 4, 5, 6, or 7 day(s). In one particularly more preferred embodiment, the dose at which the birnavirus is to be administered amounts to ≥10⁶ infectious units and <10⁷ infectious units per day for no more than/for a maximum of 1 week, wherein the dose is to be administered within 1 week after the first sign(s)/symptom(s) of a disease caused by a nidovirus such as SARS-CoV-2, e.g. after 1, 2, 3, 4, 5, 6, or 7 day(s). In one particularly even more preferred embodiment, the dose at which the birnavirus is to be administered amounts to ≥10⁶ infectious units and <10⁷ infectious units once (also dividable into two portions), ≥10⁶ infectious units and <10⁷ infectious units twice within 48 hours, preferably 24 hours, or ≥10⁶ infectious units and <10⁷ infectious units thrice within 72 hours, wherein the dose is to be administered within 1 week after the first sign(s)/symptom(s) of a disease caused by a nidovirus such as SARS-CoV-2, e.g. after 1, 2, 3, 4, 5, 6, or 7 day(s). In one particularly still even more preferred embodiment, the dose at which the birnavirus is to be administered amounts to ≥10⁶ infectious units and <10⁷ infectious units once, wherein the dose is to be administered within 1 week after the first sign(s)/symptom(s) of a disease caused by a nidovirus such as SARS-CoV-2, e.g. after 1, 2, 3, 4, 5, 6, or 7 day(s). Preferably, the above described birnavirus is an Infectious Bursal Disease Virus (IBDV) and the disease caused by a nidovirus is a severe acute respiratory syndrome (SARS) such as COVID-19. More preferably, the above described birnavirus is an Infectious Bursal Disease Virus (IBDV) and the disease caused by a nidovirus is a mild or moderate form of a severe acute respiratory syndrome (SARS) such as COVID-19. In other words: More preferably, the above described birnavirus is an Infectious Bursal Disease Virus (IBDV) and the disease caused by a nidovirus is a severe acute respiratory syndrome (SARS) such as COVID-19 not associated with an acute respiratory distress syndrome.

In a seventh aspect, the present invention relates to a birnavirus for use as medicament, wherein the birnavirus is to be administered to a subject orally, nasally, or by inhalation.

In one preferred embodiment, the birnavirus is selected from the group consisting of an avibirnavirus, an aquabirnavirus, a blosnavirus, a dronavirus, an entomobirnavirus, a ronavirus, and a telnavirus. In one more preferred embodiment, the avibirnavirus is an Infectious Bursal Disease Virus (IBDV). In one even more preferred embodiment, the IBDV is an IBDV of strain 903/78. In one still even more preferred embodiment, the IBDV of strain 903/78 comprises a nucleotide sequence according to SEQ ID NO: 1, a fragment thereof or a sequence having at least 80% sequence identity thereto, and/or a nucleotide sequence according to SEQ ID NO: 2, a fragment thereof or a sequence having at least 80% sequence identity thereto.

In particular, the nucleotide sequence can be selected from the group consisting of
(i) a nucleotide sequence according to SEQ ID NO: 1 or SEQ ID NO: 2,
(ii) a nucleotide sequence that is a fragment of the nucleotide sequence according to (i), and
(iii) a nucleotide sequence that has at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

In one further preferred embodiment, the birnavirus is a live and/or attenuated birnavirus. The birnavirus may be a naturally occurring or non-naturally occurring (live and/or attenuated) birnavirus. The non-naturally occurring birnavirus is preferably a recombinant birnavirus. The non-naturally occurring birnavirus is preferably a mutated birnavirus or a chemically treated birnavirus (e.g. treated with a protease).

The subject may be a mammal selected from the group consisting of a human being, dog, cat, sheep, goat, cow, horse, camel and pig. It is particularly preferred that the mammal is a human being.

As to the other preferred embodiments, it is referred to the first aspect of the present invention.

In an eighth aspect, the present invention relates to a pharmaceutical composition comprising a birnavirus as defined in the first aspect or a combination as defined in the second aspect, wherein the composition is in a form suitable for oral administration, nasal administration, or administration by inhalation.

In one preferred embodiment, the birnavirus is selected from the group consisting of an avibirnavirus, an aquabirnavirus, a blosnavirus, a dronavirus, an entomobirnavirus, a ronavirus, and a telnavirus. In one more preferred embodiment, the avibirnavirus is an Infectious Bursal Disease Virus (IBDV). In one even more preferred embodiment, the IBDV is an IBDV of strain 903/78. In one still even more preferred embodiment, the IBDV of strain 903/78 comprises a nucleotide sequence according to SEQ ID NO: 1, a fragment thereof or a sequence having at least 80% sequence identity thereto, and/or a nucleotide sequence according to SEQ ID NO: 2, a fragment thereof or a sequence having at least 80% sequence identity thereto.

In particular, the nucleotide sequence can be selected from the group consisting of
(i) a nucleotide sequence according to SEQ ID NO: 1 or SEQ ID NO: 2,
(ii) a nucleotide sequence that is a fragment of the nucleotide sequence according to (i) and
(iii) a nucleotide sequence that has at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

Preferably, the birnavirus is a live and/or attenuated birnavirus. The birnavirus may be a naturally occurring or non-naturally occurring (live and/or attenuated) birnavirus. The non-naturally occurring birnavirus is preferably a recombinant birnavirus. The non-naturally occurring birnavirus is preferably a mutated birnavirus or a chemically treated birnavirus (e.g. treated with a protease).

In one further preferred embodiment, the composition is in the form of a spray, an aerosol, tablet, dragee, capsule, solution, or suspension.

The pharmaceutical composition is for administration to a subject. The subject may be a mammal selected from the group consisting of a human being, dog, cat, sheep, goat, cow, horse, camel and pig. It is particularly preferred that the mammal is a human being.

As to the other preferred embodiments, it is referred to the first aspect of the present invention.

In a ninth aspect, the present invention relates to a (an in vitro) method for producing the birnavirus comprising the following steps:
(i) infecting a cell with a birnavirus,
(ii) culturing the birnavirus in the infected cell, and
(iii) isolating the cultured birnavirus or a subset of the cultured birnavirus from the infected cell.

Preferably, the cell is an immortalized cell, in particular an immortalized cell line, such as an immortalized avian cell line, e.g. the duck cell line AGE1.CR or AGE1.CR.PIX.

Thus, in an example, the method for producing the birnavirus comprises the following steps:
(i) infecting an immortalized avian cell (line) with a birnavirus,
(ii) culturing the birnavirus in the infected immortalized avian cell (line), and
(iii) isolating the cultured birnavirus or a subset of the cultured birnavirus from the infected immortalized avian cell (line).

In a specific example, the immortalized avian cell line is the duck cell line AGE1.CR or AGE1.CR.PIX.

The cell lines AGE1.CR and AGE1.CR.PIX are commercially available from ProBioGen AG. The cell line AGE1.CR.PIX was deposited with the DSMZ-Deutsche Sammlung von Mikroorganismen and Zellkulturen GmbH, Mascheroder Weg 1b, 38124 Braunschweig, Germany on Nov. 24, 2005 under accession number DSM ACC2749.

In one preferred embodiment, the birnavirus is selected from the group consisting of an avibirnavirus, an aquabirnavirus, a blosnavirus, a dronavirus, an entomobirnavirus, a ronavirus, and a telnavirus. In one more preferred embodiment, the avibirnavirus is an Infectious Bursal Disease Virus (IBDV). In one even more preferred embodiment, the IBDV is an IBDV of strain 903/78. In one still even more preferred embodiment, the IBDV of strain 903/78 comprises a nucleotide sequence according to SEQ ID NO: 1, a fragment thereof or a sequence having at least 80% sequence identity thereto, and/or

a nucleotide sequence according to SEQ ID NO: 2, a fragment thereof or a sequence having at least 80% sequence identity thereto.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### BRIEF DESCRIPTION OF THE FIGURES

The following FIGURES are merely illustrative of the present invention and should not be construed to limit the scope of the invention as indicated by the appended claims in any way.

FIG. 1: Induction of interferons in the human cell line A549 by infection with R903/78. Interferon beta, gamma and lambda induction as well as induction of genes downtstream of typel IFNs ISG56 and MxA after infection with IBDV are determined comparing mRNA levels of the respective genes before and 32 h after infection at various multiplicities of infection (MOI).

### EXAMPLES

The examples given below are for illustrative purposes only and do not limit the invention described above in any way.

### Example 1: IBDV Rescue

Both plasmids encoding the IBDV strain R903/78 segments A (SEQ ID NO: 1) and B (SEQ ID NO: 2) were transfected into AGE1.CR.PIX cells (seeded in one well of a 6 well plate the day before) using the transfection reagent Effectene (Qiagen). Five days post transfection cells (with supernatant) were lysed by 3× freeze/thaw cycles and the lysate (1 ml) was passaged onto newly seeded AGE1.CR.PIX cells (in a T25 flask). Upon the third (identically performed) passage the TCID50 virus titer and the IBDV RNA copy numbers were quantified leading to a virus titer of 2.15×10⁶ TCID50/ml and 1.2×10⁸ IBDV copies as detected by digital droplet PCR (ddPCR). Oligonucleotides used for the ddPCR: IBDV_f: 5′-TCACTACACACTGCAGAGCA-3′ (SEQ ID NO: 3); IBDV_r: 5′-GAGACTCCGACTCACTAGCC-3′ (SEQ ID NO: 4) and the IBDV Taqman probe: IBDV p: 6FAM-TGCCCAGAACCTACCGGCCA (SEQ ID NO: 5)-BBQ-3′.

### Example 2: Manufacture of R903/78 on the AGE1.CR.PIX Cell Line

For manufacturing of IBDV R903/78, the permanent immortal cell line AGE1.CR.PIX is selected for its high permissivity and titers exceeding other virus production cell lines by 100-1000fold reaching levels between 10⁹ and 10¹¹ infectious units.

One vial (1.5×10⁷ cells) was thawed, transferred to a shake flask grown in chemically defined culture medium CD-U5 with the addition of 10 ng/ml longR3IGF at 37° C. 5% CO₂ to a cell density of 8×10⁶/ml. For expansion, cell were diluted to 8×10⁵ cells/ml and transferred to a larger vessel grown to 8×10⁶/ml and finally seeded into the bioreactor used for infection (S.U.B, XDR disposable Stirred Tank Bioreactor, orbital shaken bioreactor) at 8×10⁵ cells/ml.
For production of IBDV R903/78 cells are infected at an MOI of 0.05 when density reaches 2×10⁶/ml. Incubation is continued without feeding for 120 h. During this incubation time cells remain intact. Virus is recovered from cell culture supernatant only without destroying infected cells. Intact cells are removed by filtration with 3 μm polypropylene filters (Sartopure PP3, Sartorius, Germany) at flow rates of 170 LMH.

Titer is determined in 96 well plates seeded with 10⁵ AGE1.CR.PIX cells/well, infected with 10× serial dilutions of the virus suspension, incubated for 72 h. Infectious titer is calculated as tissue culture infection dose 50 (TCID50) using the Spearman-Karber-algorithm. The virus suspension is diluted to a final concentration of 2×10⁶, 2×10⁷, 2×10⁸, 2×10⁹ with the carbohydrate-based, stabilizing buffer (10 mM Tris base, pH 7.2 at RT, 75 mM NaCl, 1 mM MgCl₂, 0.0025% Polysorbate 80 containing 15% (w/v) sucrose). Final virus suspension was subjected to sterile filtration applying a single use 0.22 μm filter with polyvinylidenfluorid (PVDF)-membrane.

### Example 3: IBDV Induces Type I and III Interferons (IFN)

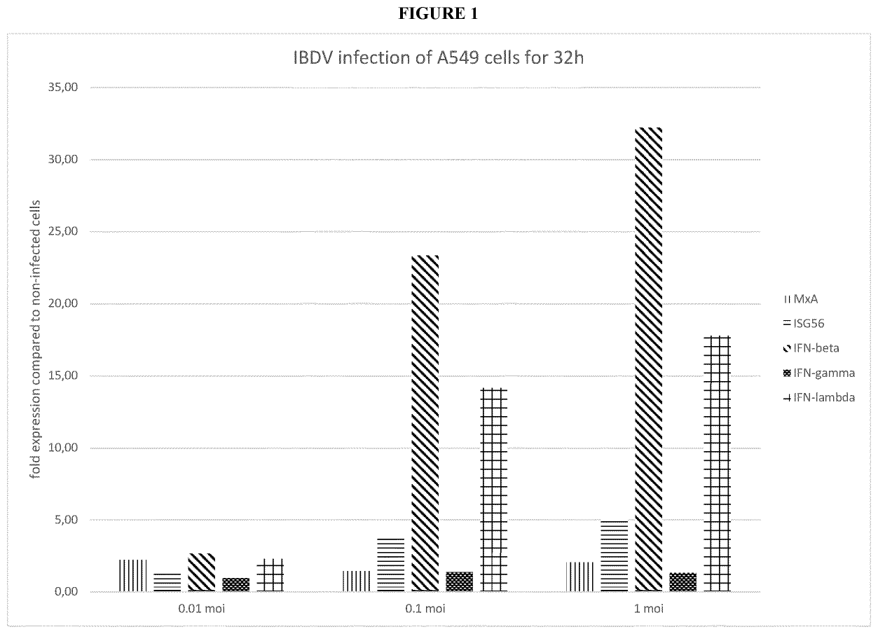

The human lung epithelial cells (A549) is typically used as an indicator cell line to analyse which type of interferons is induced by a specific virus. A549 cells are fully susceptible to infection with IBDV. To study whether type I (IFN-alpha IFN-beta), type II (IFN-gamma) and type III (IFN-lambda) are induced by IBDV R903/78, A549 cells were infected at different MOIs (0.01, 0.1 and 1) for 32 h. Subsequently, RNA was harvested out of the infected cells using the innuPREP Virus RNA kit (Analytik Jena) including a DNase digestion step. The expression levels of the different interferons (IFN-beta, IFN-gamma, IFN-lambda) and interferon-regulated genes (human myxovirus resistance protein-A (MxA) and the human interferon-stimulated gene 56 (ISG56)) were analysed by a quantitative one-step RT-PCR using the GoTaq® 1-Step RT-qPCR kit (Promega) and the following oligonucleotides: IFN-beta_f (5′-GACGCCGCATTGACCATCTA-3′ SEQ ID NO: 6), IFN-beta-r (5′-CCTTAGGATTTCCACTCTGACT-3′ SEQ ID NO: 7), IFN-lambda_f (5′-TGAGCTGGCCCTGACGCTGAA-3′ SEQ ID NO: 8), IFN-lambda_r (5′-AGGCGGAGGTTGAAGGTGA-3′ SEQ ID NO: 9), IFN-gamma_f (5′-GAAAAGCTGACTAATTATTCGGTAACTG-3′ SEQ ID NO: 10), IFN-gamma_r (5′-GTTCAGCCATCACTTGGATGAG-3′ SEQ ID NO: 11), ISG56_f (5′-CCTGGAGTACTATGAGCGGGC-3′ SEQ ID NO: 12), IFG56_r (5′-TGGGTGCCTAAGGACCTTGTC-3′ SEQ ID NO: 13), MxA_f (5′-AGGTCAGTTACCAGGACTAC-3′ SEQ ID NO: 14) and MxA_r (5′-ATGGCATTCTGGGCTTTATT-3′ SEQ ID NO: 15). The relative expression levels were normalized against non-infected cells. Glyceraldehyde-3-phosphate dehydrogenase (GAPDH) was used as an internal standard: GAPDH specific oligonucleotides GAPDH_f (5′-GGTATCGTGGAAGGACTCATGAC-3′ SEQ ID NO: 16) and GAPDH_r (5′-ATGCCAGTGAGCTTCCCGTTCAG-3′ SEQ ID NO: 17). As shown in FIG. 1, IBDV strongly induces IFN-beta in cells infected for 32 h with IBDV at multiplicity of infection 1 (MOI1) (about 30×) and IFN lambda (5-10×). Surprisingly, in contrast to other viruses (Adenovirus, Encephalomyocarditis virus) IFN gamma is not induced in A549 cells. Genes downstream of IFN are not yet strongly induced, likely due to an early harvest time.

IFNs are the most potent innate protection when we are immunologically naïve and this response is declining rapidly in aged people. Upon receptor binding IFNs stimulate various ISGs that inhibit virus replication. They shut off viral RNA translation, promote of mRNA degradation, block virus entry and egress tethering viruses to the cell surface and block autophagy. Autophagy mechanisms are hijacked by viruses from various genera including Nidoviuses to reorganise membranes for virus replication.

Nidoviruses are extremely sensitive to type I IFNs. For instance, in Vero cells, IFN alpha treatment reduces SARS-CoV-2 titers by 3-4 orders (Lokugamage, K. G., Schindewolf, C., Menachery, V. D. 2020. SARS-CoV-2 sensitive to type I interferon pretreatment. bioRxiv, 2020.03.07.982264.) which makes IBDV R903/78's interferon activation properties promising to counter nidovirus infections.

Interferons treatment, in particular extended treatment is associated with severe side effects. Higher toxicity of IFN gamma compared to IFN alpha and beta limits its clinical use. While IFN-lambda is as potent inducing an innate response against viruses it shows least side effects. Therefore, the natural specific induction profile maximizes antiviral action with minimal side effects opening the therapeutic window.

### Example 4: Case Histories in Human Patients

### Case 1

A 43 years old male patient reported extreme muscle pains, shivering, tiredness, and headaches. While the pains almost disappeared by the 3rd day, constant headache and dizziness, and very low energy to move around remained. When the patient would stand up felt very dizzy, almost ready to lose his balance. By the end of the first week of symptoms, the patient complained about a decrease in his ability to smell odours in and around the house. Six days after the first symptoms a government-sponsored COVID-19 PCR test proved to be positive. Then, oral treatment with the IBDV strain started with 10⁶ infectious units of the virus in the evening, after dinner. By the next day morning, the patient reported that his headaches have lessened, and the dizziness and unsteadiness when standing up has also been less prominent. Overall, there was a higher level of motivation to do physical activity, like walking in the house and moving around. The second oral IBDV with 10⁶ infectious units of the virus was again administered the next day in the evening, after dinner. The patient reported that after the second vial treatment, he had a very good night of sleep, for the first time without any symptoms, or pains, with no headaches and or muscle aches. By morning he woke up with more energy without dizzy feelings or headaches. He was able to sit in front of the computer and read emails, articles, think, and write without any problems. Before the viral treatment, all these activities were difficult, as he had felt a loss of short-term memory and a really difficult time to concentrate on an intellectual task, as simple as just paying an invoice online. Also, after the second treatment dose, he felt motivated to do some minor physical exercise, like slow and easy stationary cycling and also light gymnastics exercising. The third oral IBDV with 10⁶ infectious units of the virus was administered the next day also in the evening, after dinner. After the 3rd viral dose, he felt more motivated physically, even able to do a more intensive home-based exercise for the first time since he started with the COVID symptoms. This would mean more moderate paced stationary cycling and light weight lifting exercises at home. One other thing to note was that his sense of smell, which deteriorated in the first few days after the first COVID symptoms appeared, came back as well. He could smell the candles in the house, already from a distance, which before he was unable to do. The only side effect the patient reported mild heartburn in his throat and in the food pipe, which lasted for three days. Three weeks after the oral IBDV therapy the patient's serum was tested for the presence of IBDV neutralizing antibodies by virus neutralization as described earlier (Hornyak, et al., 2015). The highest serum dilution that inhibited plaque formation was found to be 1/32 demonstrating the presence of anti-IBDV antibodies. This confirmed the results obtained in an animal model that following oral administration of IBDV neutralizing antibodies were induced. Considering that humans are not a natural host of this virus, the appearance of neutralizing antibodies after oral administration is not trivial. To our best knowledge, this is the first early COVID-19 patient with mild symptoms who was successfully treated by IBDV therapy.

### Case 2

A 40 years old female medical assistant had no fever at all but felt strong joint pains and sharp burning pain in her right chest with increasing dyspnea and severe weakness. She had a positive COVID (antigen detect) test three days later, she stayed at home, separated from her family. Two doses of the IBDV strain, 5×10⁵ infective units each, were orally administered within 12 h (first dose in the morning, the second in evening). She got significantly better by the next day, therefore, she hesitated whether to have a third dose and finally decided not to continue treatment. She had a control COVID test 8 days later, which was negative. She is completely free of any symptoms or complaints since.

1. A birnavirus for use in the treatment of a disease caused by a nidovirus.

2. The birnavirus for use of claim 1, wherein the birnavirus is an avibirnavirus, an aquabirnavirus, a blosnavirus, a dronavirus, an entomobirnavirus, a ronavirus, or a telnavirus.

3. The birnavirus for use of claim 2, wherein the avibirnavirus is an Infectious Bursal Disease Virus (IBDV).

4. The birnavirus for use of claim 3, wherein the IBDV is an IBDV of strain 903/78.

5. The birnavirus for use of claim 4, wherein the IBDV of strain 903/78 comprises a nucleotide sequence according to SEQ ID NO: 1, a fragment thereof or a sequence having at least 80% sequence identity thereto, and/or a nucleotide sequence according to SEQ ID NO: 2, a fragment thereof or a sequence having at least 80% sequence identity thereto.

6. The birnavirus for use of any one of claims 1 to 5, wherein the birnavirus is a live birnavirus.

7. The birnavirus for use of any one of claims 1 to 6, wherein the birnavirus is a naturally occurring or non-naturally occurring birnavirus.

8. The birnavirus for use of claim 7, wherein the non-naturally occurring birnavirus is a recombinant birnavirus.

9. The birnavirus for use of any one of claim 7 or 8, wherein the non-naturally occurring birnavirus is a mutated birnavirus.

10. The birnavirus for use of any one of claims 7 to 9, wherein the non-naturally occurring birnavirus is a chemically treated birnavirus.

11. The birnavirus for use of any one of claims 1 to 10, wherein the nidovirus is a virus of the family of Coronaviridae, Arteriviridae, Roniviridae, or Mesoniviridae.

12. The birnavirus for use of claim 11, wherein the virus of the family of Coronaviridae is selected from the group consisting of MERS-CoV, SARS-CoV, SARS-CoV-2, a coronavirus that gains entry by the ACE2 receptor, and an Infectious Bronchitis Virus (IBV).

13. The birnavirus for use of claim 11, wherein the virus of the family of Arteriviridae is selected from the group consisting of a virus that causes porcine reproductive and respiratory syndrome (PRRS) and a virus that causes equine viral arteritits (EVA).

14. The birnavirus for use of any one of claims 1 to 13, wherein the birnavirus is for administration to a subject receiving or having received at least one further therapy against a disease caused by a nidovirus.

15. The birnavirus for use of claim 14, wherein the at least one further therapy against a disease caused by a nidovirus is selected from the group consisting of an antiviral drug therapy, an antibacterial drug therapy, an anti-inflammatory drug therapy, antifungal drug therapy, an antipyretic agent therapy, and corticoid therapy.

16. The birnavirus for use of claim 15, wherein
(i) the antiviral drug is selected from the group consisting of chloroquine, remdesivir, darunavir, favipiravir, lopinavir, and ritonavir, or is a combination thereof,
(ii) the antibacterial drug is selected from the group consisting of ceftriaxone, amoxicillin/clavulanic acid, piperacillin/tazobactam, and azithromycin, or is a combination thereof,
(iii) the anti-inflammatory drug is selected from the group consisting of ibuprofen and metamizole,
(iv) the antifungal drug is selected from the group consisting of voriconazole and isavuconazole,
(v) the antipyretic agent is paracetamol, and/or
(vi) the corticoid is dexamethasone.

17. The birnavirus for use of any one of claims 14 to 16, wherein the subject is a mammal selected from the group consisting of a human being, dog, cat, sheep, goat, cow, horse, camel and pig.

18. The birnavirus for use of any one of claims 1 to 17, wherein the dose at which the birnavirus is to be administered amounts to
≥10⁶ infectious units and <10⁷ infectious units once, or
≥10⁶ infectious units and <10⁷ infectious units twice within 48 hours, preferably 24 hours.

19. A combination comprising at least one birnavirus and at least one further active agent for use in the treatment of a disease caused by a nidovirus.

20. The combination for use of claim 19, wherein the nidovirus is a virus of the family of Coronaviridae, Arteriviridae, Roniviridae, or Mesoniviridae.

21. The combination for use of claim 20, wherein the virus of the family of Coronaviridae is selected from the group consisting of MERS-CoV, SARS-CoV, SARS-CoV-2, a coronavirus that gains entry by the ACE2 receptor, and an Infectious Bronchitis Virus (IBV).

22. The combination for use of claim 20, wherein the virus of the family of Arteriviridae is selected from the group consisting of a virus that causes porcine reproductive and respiratory syndrome (PRRS) and a virus that causes equine viral arteritits (EVA).

23. The combination for use of any one of claims 19 to 22, wherein the at least one further active agent is selected from the group consisting of an antiviral drug, an antibacterial drug, an anti-inflammatory drug, antifungal drug, an antipyretic agent, and a corticoid.

24. The combination for use of claim 23, wherein
(i) the antiviral drug is selected from the group consisting of chloroquine, remdesivir, darunavir, favipiravir, lopinavir, and ritonavir, or is a combination thereof,
(ii) the antibacterial drug is selected from the group consisting of ceftriaxone, amoxicillin/clavulanic acid, piperacillin/tazobactam, and azithromycin, or is a combination thereof,
(iii) the anti-inflammatory drug is selected from the group consisting of ibuprofen and metamizole,
(iv) the antifungal drug is selected from the group consisting of voriconazole and isavuconazole,
(v) the antipyretic agent is paracetamol, and/or
(vi) the corticoid is dexamethasone.

25. The combination for use of any one of claims 19 to 24, wherein the dose at which the birnavirus is to be administered amounts to
≥10⁶ infectious units and <10⁷ infectious units once, or
≥10⁶ infectious units and <10⁷ infectious units twice within 48 hours, preferably 24 hours.

26. The combination for use of any one of claims 19 to 25, wherein the at least one birnavirus and the at least one further active agent are to be administered concurrently or consecutively.

27. The combination for use of any one of claims 19 to 26, wherein the combination comprises different birnaviruses.

28. The combination for use of claim 27, wherein the different birnaviruses are birnaviruses of different types or from different strains.

29. A pharmaceutical composition comprising a birnavirus as defined in any one of claims 1 to 18 or a combination as defined in any one of claims 19 to 28 for use in the treatment of a disease caused by a nidovirus.

30. The pharmaceutical composition for use of claim 29, wherein the composition further comprises one or more pharmaceutical acceptable excipient(s), diluent(s), and/or carrier(s).

31. The pharmaceutical composition for use of claim 29 or 30, wherein the pharmaceutical composition is in a form suitable for oral administration, nasal administration, or administration by inhalation.

32. A reservoir comprising a birnavirus as defined in any one of claims 1 to 18, a combination as defined in any one of claims 19 to 28, or a pharmaceutical composition as defined in any one of claims 29 to 31, wherein the reservoir is designed to be inserted into an oral or a nasal applicator.

33. An oral or a nasal applicator comprising
(i) a reservoir comprising a birnavirus as defined in any one of claims 1 to 18, a combination as defined in any one of claims 19 to 28, or a pharmaceutical composition as defined in any one of claims 29 to 31, and
(ii) a mouth piece or nose piece, wherein the mouth piece or the nose piece is connected with/attached to the reservoir.

34. The oral or nasal applicator of claim 33, wherein the mouth piece or nose piece is designed so as to allow complete insertion into the mouth or nose.

35. A kit comprising
(i) a packaging material, and
(ii) a birnavirus as defined in any one of claims 1 to 18, a combination as defined in any one of claims 19 to 28, a pharmaceutical composition as defined in any one of claims 29 to 31, a reservoir of claim 32, or an oral or nasal applicator of claim 33 or 34.

36. The kit of claim 35, wherein the kit further comprises
(iii) a label or packaging insert contained within the packaging material indicating that subjects receiving the birnavirus, the combination or the pharmaceutical composition can be treated for a disease caused by a nidovirus.

37. The kit of claim 36, wherein the label or packaging insert further comprises the information that the dose at which the birnavirus is to be administered amounts to ≥10⁶ infectious units and <10⁷ infectious units once, or ≥10⁶ infectious units and <10⁷ infectious units twice within 48 hours, preferably 24 hours.

38. A birnavirus for use as medicament, wherein the birnavirus is to be administered to a subject orally, nasally, or by inhalation.

39. A pharmaceutical composition comprising the birnavirus as defined in any one of claims 1 to 18 or the combination as defined in any one of claims 19 to 28, wherein the composition is in a form suitable for oral administration, nasal administration, or administration by inhalation.

40. The pharmaceutical composition of claim 39, wherein the composition is in the form of a spray, an aerosol, tablet, dragee, capsule, solution, or suspension.
